(19) United States
(12) Patent Application Publication
(10) Pub. No.: US 2024/0307561 A1
(43) Pub. Date: Sep. 19, 2024

(54) RADIOLABELED PARP INHIBITOR CONJUGATES FOR CANCER TREATMENT

(71) Applicant: DUKE UNIVERSITY, DURHAM, NC (US)

(72) Inventors: MICHAEL ROD ZALUTSKY, DURHAM, NC (US); GANESAN VAIDYANATHAN, DURHAM, NC (US)

(73) Assignee: DUKE UNIVERSITY, DURHAM, NC (US)

(21) Appl. No.: 18/575,098

(22) PCT Filed: Jun. 29, 2022
(86) PCT No.: PCT/US22/35542

§ 371 Date: Dec. 28, 2023

Related U.S. Application Data
(60) Provisional application No. 63/216,301, filed on Jun. 29, 2021.
(60) Provisional application No. 63/355,336, filed on Jun. 24, 2022.

(51) Int. Cl. A61K 51/04; A61K 45/06; A61K 51/10

(52) U.S. Cl. CPC A61K 51/0459; A61K 45/06; A61K 51/1045

(57) ABSTRACT

PARP inhibitor conjugates, including radiolabeled PARP inhibitor conjugates, and methods of treating cancer using the same.

### RELATED APPLICATIONS

This application claims priority to U.S. Provisional Application Ser. No. 63/216,301, filed Jun. 29, 2021, and U.S. Provisional Application Ser. No. 63/355,336, filed Jun. 24, 2022, both of which are incorporated herein by reference.

### TECHNICAL FIELD

The present invention generally relates to poly(ADP-Ribose) polymerase inhibitor (PARPi) conjugates for therapy, and more particularly, PARPi conjugates that include a tumor targeting agent, such as radiolabeled PARPi conjugates that include a tumor targeting agent.

### BACKGROUND

Poly(ADP-ribose) polymerase (PARP) is a family of enzymes involved in DNA repair, replication, and gene transcription. In particular, PARP-1 acts as a first responder that detects DNA damage and is implicated in several repair pathways. Based upon this molecular function and the observation that PARP-1 is overexpressed in a number of tumors compared to normal tissues, PARP-1 inhibitors (PARPi) have been investigated extensively for the treatment of various cancers. The idea of combining PARP-1 inhibitors with radioactivity has also been explored because PARP-1 binding to DNA has the potential to increase the efficiency of cell killing following release of the decay energy in cancer cell nuclei.

Notwithstanding the keen interest in PARP-1 inhibitors, recent studies show only modest efficacy. Accordingly, there is a need in the art for further improvements to PARPi therapies.

### SUMMARY OF THE INVENTION

The present application provides conjugates comprising: (i) a tumor specific targeting agent, (ii) a radiolabeled PARP inhibitor, such as a radiolabeled PARP-1 inhibitor, and (iii) a linker between the targeting agent and the PARP inhibitor. In specific embodiments contemplated herein, the linker allows escape of the radiolabeled PARP inhibitor from the lysosome/endosome after receptor-mediated internalization so it can move to the cytoplasm and reach the nucleus where it can bind to PARP. The disclosed PARP inhibitor conjugates can selectively target PARP over-expressing cells, potentially lowering serum levels necessary to achieve desired therapeutic benefits, and concomitantly, ameliorating or avoiding side effects associated with systemic administration of small molecule PARP inhibitors. In a preferred aspect of the invention, the conjugates include a radionuclide that is suitable for radiotherapy of the targeted cancer cells.

In an embodiment, a composition comprises a radiolabeled poly(ADP-ribose) polymerase-1 inhibitor (PARPi(Rd)) conjugate, wherein the conjugate comprises PARPi(Rd) coupled to a tumor targeting agent (TTA).

In an embodiment, the PARPi conjugate comprises Formula I:

PARPi(Rd)-L-TTA; (I)
wherein Rd is a radiolabel and PARPi(Rd) is a radiolabeled PARPi,
wherein L is a linker and TTA is a tumor targeting agent, and
wherein the L couples the PARPi(Rd) to the TTA.

In a specific embodiment, the PARP inhibitor is a PARP-1 inhibitor. Examples of PARP inhibitors include, but are not limited to: olaparib, veliparib, rucaparib, niraparib, pamiparib, EB-47, and talazoparib. A PARP inhibitor may be selected to increase PARP-1 binding affinity to DNA compared to the binding affinity to DNA of uninhibited PARP-1. A PARP inhibitor may be selected to slow the release of PARP-1 from DNA compared to the release of uninhibited PARP-1 from DNA. In a specific embodiment, the PARP inhibitor comprises olaparib. In another specific embodiment, the PARP inhibitor comprises talazoparib.

In a specific embodiment, the TTA is a single domain antibody fragment (sdAb). In another specific embodiment, the TTA is a prostate specific membrane antigen (PSMA) inhibitor/ligand.

In a specific embodiment, Rd comprises a radionuclide that emits short-range radiation. In another specific embodiment, Rd decays by the emission of Auger electrons and/or alpha particles.

In a specific embodiment, Rd has the structure:

Cg¹-ArQ-Cg²;
wherein Cg¹ is a coupling group that couples ArQ to the PARPi, and Cg² is a coupling group that couples ArQ to the L;
wherein Cg¹ and Cg² are each, independently, succinimidyloxycarbonyl; maleimide;
—OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or
   Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
   wherein R¹ and R² are independently selected from H, branched or unbranched C₁₋₁₀ alkyl, or aryl;
wherein Ar is an aryl, heteroaryls or a radionuclide chelating agent; and
wherein Q is radionuclide.
   In a specific embodiment, L has the structure:

ES¹-SS-ES²
wherein ES¹ has the structure: Cg³-SP-;
wherein SS is a disulfide bond;
wherein ES² has the structure: -SP-Cg⁴-;
wherein Cg³ and Cg⁴ are each, independently, succinimidyloxycarbonyl, maleimide,
—OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein SP (spacer) is —C₁-C₁₀ alkylene-; —C₁-C₁₀ alkenylene-; —C₁-C₁₀ alkynylene-; carbocyclo-; —O—(C₁-C₈ alkylene)-; O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-;
-(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—,
   wherein r is an integer ranging from 1-10;
wherein Cg³ is a coupling group that forms a covalent bond to the Rd; and wherein Cg⁴ is a coupling group that forms a covalent bond to the TTA.

In a specific embodiment, L has the structure:

ES-ECL-SIG
wherein ES has the structure: Cg³-SP-Cg⁴;
Cg³ and Cg⁴ are each, independently, succinimidyloxycarbonyl, maleimide, —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein Cg³ is a coupling group that forms a covalent bond to the Rd component and Cg⁴ is a coupling group that forms a covalent bond to ECL;
   wherein SP is —C₁-C₁₀ alkylene-; —C₁-C₁₀ alkenylene-; —C₁-C₁₀ alkynylene-; carbocyclo-; —O—(C₁-C₈ alkylene)-; O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-;
-(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—, wherein r is an integer ranging from 1-10;
wherein ECL is an enzyme cleavable linker; and
wherein SIG is a self-immolative group that forms a covalent bond between the ECL and the TTA, wherein upon cleavage of the CL, the SIG decomposes to release the TTA from the ECL.

In a specific embodiment, the ECL is cleavable by a lysosomal enzyme. In a specific embodiment, the lysosomal enzyme may be a lysosomal protease. In a specific embodiment, the ECL is a peptide composed of two to four amino acids. In another specific embodiment, the lysosomal enzyme is lysosomal glycosidase.

In an embodiment, the PARPi(Rd)-L has the structure (VI):

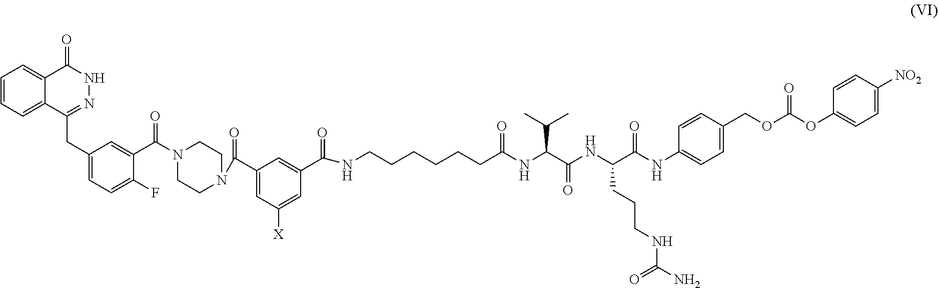

where X is a radionuclide.
In an embodiment, the PARPi(Rd)-L has the structure (IX):

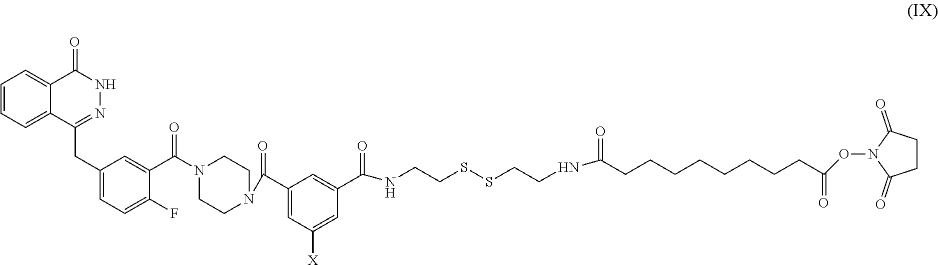

where X is a radionuclide.
In an embodiment, the PARPi(Rd)-L has the structure (13):

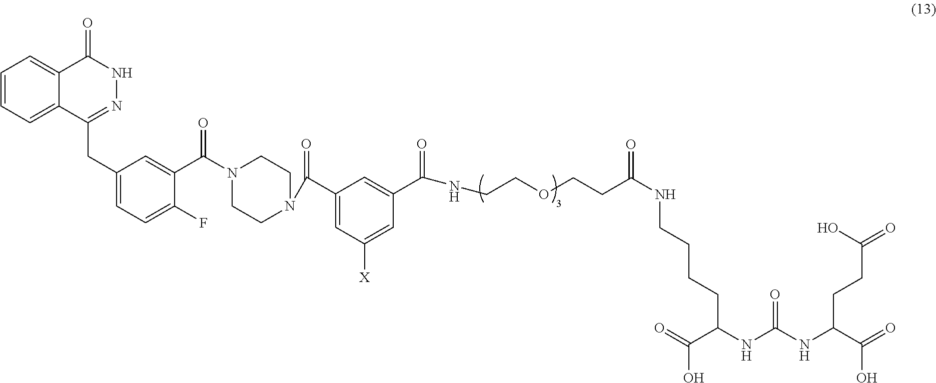

where X is a radionuclide.
In an embodiment, the PARPi(Rd)-L has the structure:

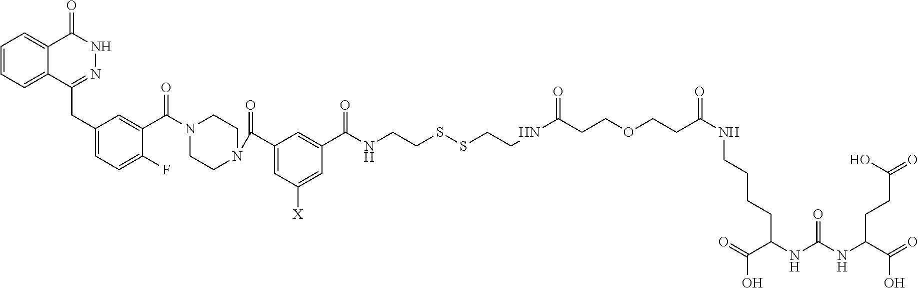

where X is a radionuclide.

In an embodiment, a composition comprises a poly(ADP-ribose) polymerase-1 inhibitor (PARPi) conjugate, wherein the conjugate comprises a PARPi coupled to a tumor targeting agent (TTA). In a specific embodiment, the PARPi conjugate comprises Formula I:

PARPi-L-TTA; (I)
wherein L is a linker and TTA is a tumor targeting agent, and
wherein the L couples the PARPi to the TTA.

In an embodiment, a pharmaceutical composition comprises a conjugate as described herein and a pharmaceutically acceptable carrier. In a specific embodiment, the pharmaceutical composition further comprises a photosensitizing agent. In an embodiment, the pharmaceutical composition can be used for cancer therapy.

In an embodiment, a method of radiosensitizing a tumor in a cancer subject comprises administering to the subject a pharmaceutical composition that comprises a conjugate as described in Formula I and a pharmaceutically acceptable carrier.

In an embodiment, a method of treating cancer in a subject comprises the steps of (i) administering to the subject a pharmaceutical composition that comprises a conjugate as described herein and a pharmaceutically acceptable carrier, and (ii) administering ionizing radiation. The method of treating cancer can be applied to ovarian cancer, breast cancer, prostate cancer, or brain cancer. In an embodiment, the subject has a deficiency in at least one gene involved in the homologous recombination repair (HRR) pathway.

In an embodiment, a method of treating cancer in a subject comprises administering to the subject a pharmaceutical composition that comprises a conjugate as described herein. In a specific embodiment, the pharmaceutical composition is administered in combination with chemotherapy. In another specific embodiment, the pharmaceutical composition is administered in combination with radiotherapy. This method of treating cancer can be applied to ovarian cancer, breast cancer, prostate cancer, or brain cancer. In an embodiment, the subject has a deficiency in at least one gene involved in the homologous recombination repair (HRR) pathway.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts uptake and internalization of (20S,24S)-1-(3-(4-(2-fluoro-5-((4-oxo-3,4-dihydrophthalazin-1-yl)methyl)benzoyl)piperazine-1-carbonyl)-5-[¹³¹I]iodophenyl)-1,14,22-trioxo-5,8,11-trioxa-2,15,21,23-tetraazahexacosane-20,24,26-tricarboxylic acid ([¹³¹I]PARP-PSMA combo agent) in PSMA⁺ PIP cells.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE INVENTION

The present invention provides novel targeted PARP inhibitor conjugates, processes for preparing the conjugates and uses of the conjugates. While it is known that PARP inhibitors, including radiolabeled PARP inhibitors, can be used in therapeutic and theranostic strategies for cancer, current radiolabeled PARP inhibitors are lipophilic and passively diffuse into both cancerous and non-cancerous cells, detracting from their therapeutic index. The present application provides compositions and methods to exploit the advantages of PARP (presence in many types of tumors, ability to facilitate delivery of short range of action therapeutics to the cell nucleus) without the disadvantages of current approaches that lack tumor specificity.

More specifically, disclosed herein are conjugates having such properties, with representative conjugates comprising: (i) a tumor specific targeting agent, (ii) a PARP inhibitor, such as a PARP-1 inhibitor, and (iii) a linker between the targeting agent and the PARP inhibitor. In specific embodiments contemplated herein, the linker allows escape of the radiolabeled molecule from the lysosome/endosome after receptor-mediated internalization so it can move to the cytoplasm and reach the nucleus where it can bind to PARP. The disclosed PARP inhibitor conjugates can selectively target PARP over-expressing cells, potentially lowering serum levels necessary to achieve desired therapeutic benefits, and concomitantly, ameliorating or avoiding side effects associated with systemic administration of small molecule PARP inhibitors.

Scientific and technical terms used in connection with the present invention, unless indicated otherwise herein, shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, radiochemistry, genetics and protein and nucleic acid chemistry and hybridization described herein are those well-known and commonly used in the art.

### I. PARPs

Poly(ADP-ribose) polymerase (PARP) proteins, also called ADP-ribosyl transferase diphtheria toxin-like enzymes (ARTDs), constitute a superfamily of enzymes implicated in a variety of cellular functions, including the cellular response to stress, notably physiological conditions that occur in the tumor microenvironment, such as hypoxia, autophagy, and cancer-associated immune responses. PARP proteins share a 50-amino acid sequence recognized as a “PARP signature” responsible for synthesis and transfer of ADP-ribose to a variety of substrates through a process called poly(ADP-ribosyl)ation or PARylation.

There are 17 members of the PARP superfamily: PARP-1 (ARTD1), PARP-2 (ARTD2), PARP-3 (ARTD3), PARP-4 (ARTD4), Tankyrase-1 (TANK1; PARP-5A, ARTD5), Tankyrase-2 (TANK2; PARP-5B, ARTD6), PARP-6 (ARTD17), PARP-7 (ARTD7, TIPARP), PARP-8 (ARTD16), PARP-9 (ARTD9, BAL1), PARP-10 (ARTD10), PARP-11 (ARTD11), PARP-12 (ARTD12), PARP-13 (ARTD13), PARP-14 (ARTD8, BAL2), PARP-15 (ARTD7, BAL3), and PARP-16 (ARTD15). The superfamily is further subdivided according to enzymatic function:
DNA-dependent PARPs, containing zinc finger DNA binding domains, and which are active during DNA damage (PARP-1, PARP-2, and PARP-3);
Tankyrases, which contain ankyrin domain repeats and sterile a motifs (SAM) responsible for protein-protein interactions (PARP-5A and PARP-5B);
CCCH PARPs, containing CCCH motifs involved in RNA binding (PARP-7, PARP-12, PARP-13);
Macro-PRPs, containing macrodomain folds, and which mediate the migration of the proteins to ADP ribosylation sites (PARP-9, PARP-14, and PARP-15); and
Other PARPs, which do not align with any of the foregoing subgroups (PARP-4, PARP-6, PARP-8, PARP-10, PARP-11 and PARP-16).

PARP-1 was the first identified PARP and has been most extensively studied. PARP-1 is encoded by region 1q41-q42 and is constitutively expressed. In the last decade, much attention has been focused on PARP-1 inhibitors, particularly for the treatment of breast cancer gene 1 or 2 (BRAC_{1/2})-deficient ovarian and breast cancers. See e.g., Jannetti et al, Frontiers in Pharmacology, 2020, 11: 170; Martí et al, Cancers, 2020, 12:739; and Demény and Virág, Cancers, 2021, 13: 2042.

PARPi conjugates of the invention may include an inhibitor of any PARP. In some aspects of the invention, PARPi conjugates comprise an inhibitor of a DNA-dependent PARP (e.g., PARP-1, PARP-2, and PARP-3). In preferred aspects of the invention, PARPi conjugates comprise an inhibitor of PARP-1. In other aspects, PARPi conjugates comprise an inhibitor of PARP-2, an inhibitor of PAPR-3, an inhibitor of PARP-5a, or an inhibitor of PARP-5b.

### II. PARP Inhibitors

A “PARP inhibitor” as used herein is any agent that diminishes or reduces PARP functions. Numerous assays of PARP enzyme function are known in the art and often commercially available. Representative assays include but are not limited to PARP1 Enzyme Activity Assay (Sigma-Aldrich 17-10149), PARP/Apoptosis Universal Colorimetric Assay Kit (R&D Systems 4677-096-K), HT PARP In Vivo Pharmacodynamic Assay II (R&D Systems 4520-096-K), PARP1 Colorimetric Activity Assay Kit (BPS Bioscience 80580), PARP1 Chemiluminescent Activity Assay Kit (Amsbio 80551), Wigle et al. Cell Chem Biol., 2020, 27:7, P877-887.E14, and Belhadj et al. Plos One, 2021, 16(1) e0245369.

In certain aspects of the invention, a PARP inhibitor will diminish or reduce PARP function at least 10%, at least 20%, at least 30%, at least 40%, at least 50%, at least 60%, at least 70%, at least 80%, at least 90%, at least 95%, or at least 99%, in comparison to a control assay performed in the absence of the PARP inhibitor. In other aspects of the invention, a PARP inhibitor will diminish or reduce PARP function at least 2-fold, at least 5-fold, at least 10-fold, at least 50-fold, at least 100-fold, or more, in comparison to a control assay performed in the absence of the PARP inhibitor.

Representative PARP-1 inhibitors include, but are not limited to, olaparib, rucaparib, niraparib, pamiparib, and talazoparib. Additional representative PARP-1 inhibitors include but are not limited to veliparib (AbbVie), NMS-P293 (Nerviano Medical Sciences), stenoparib (Allarity Therapeutics), JPI-289 (Jeli Pharmaceuticals), NOV-140101 (Ildong Pharmaceutical), SC-10914 (Jiangxi Qingfeng Pharmaceutical), senaparib (Impact Therapeutics), fluzoparib (Jiangsu Hengrui Medicine), pamiparib (BeiGene) and 1-piperazineacetamide-4-[1-(6-amino-9H-purin-9-yl)-1-deoxy-d-ribofuranuron]-N-(2,3-dihydro-1H-isoindol-4-yl)-1-one (EB-47) (Qiu et al. Acta Cryst. 2014; D70 2740-2753, which is incorporated herein by reference). See e.g., U.S. Pat. Nos. 7,151,102, 10,130,636, 8,071,579, 8,012,976, 9,580,410, 8,993,594, 8,236,802, 8,815,891, 9,844,550, 9,926,304, 9,273,052, 9,617,273, and US Published Application No. 2020/0030339, each of which is incorporated herein in its entirety.

Additional contemplated PARP-1 inhibitors useful in the invention also include derivatives of any of the afore mentioned inhibitors that have substantially similar activity, e.g., at least 50% inhibition, or at least 60% inhibition, or at least 70% inhibition, or at least 80% inhibition, or at least 90% inhibition, or at least 95% inhibition, or at least 99% inhibition, as compared to the named inhibitor. The above-identified PARP-1 inhibitors may also be used as inhibitors of other PARPs.

For radiolabeled PARPi, it is desirable to get the radionuclide close to the DNA and maintain localization of the radiolabeled PARPi in the vicinity of the DNA. This may be achieved by selecting PARPi inhibitors that promote significant and prolonged binding of PARP-1 to DNA. PARP inhibitors may be classified based on how the binding of the PARPi to PARP-1 influences the binding affinity and the rate of release of PARP-1 from DNA. (See Zandarashvili et al. “Structural basis for allosteric PARP-1 retention on DNA breaks” Science 368:46, 2020, which is incorporated herein by reference). Type I inhibitors cause a relatively large increase in PARP-1 DNA binding affinity and a slow release of PARP-1 from DNA, compared to uninhibited PARP-1. Type I inhibitors exhibit an apparent equilibrium binding affinity (K_{D}) of less than about 25 nM and/or a dissociation rate constant (k_{d}) of less than about 1.52×10⁻³ s⁻¹. Examples of Type I PARP inhibitors include 1-piperazineacetamide-4-[1-(6-amino-9H-purin-9-yl)-1-deoxy-d-ribofuranuron]-N-(2,3-dihydro-1H-isoindol-4-yl)-1-one (EB-47) and benzamide adenine dinucleotide (BAD). Type II inhibitors cause a small increase in PARP-1 DNA binding affinity and a slightly slower release of PARP-1 from DNA, compared to uninhibited PARP-1. Type II inhibitors exhibit an apparent equilibrium binding affinity (K_{D}) of between about 25 nM and 100 nM and/or a dissociation rate constant (k_{d}) of between about 1.5×10⁻³ s⁻¹ and 3.0×10⁻³ s⁻¹. Examples of Type II PARP inhibitors include olaparib and talazoparib. Finally, Type III PARP inhibitors are characterized by causing a decrease in PARP-1 DNA binding affinity and a faster release of PARP-1 from DNA, compared to uninhibited PARP-1. Type III inhibitors exhibit an apparent equilibrium binding affinity (K_{D}) of greater than about 100 nM and/or a dissociation rate constant (k_{d}) of greater than 3.5×10⁻³ s⁻¹. Examples of Type III PARP inhibitors include veliparib, rucaparib and niraparib.

In an aspect of the invention, a PARPi used in a PARPi conjugate is a Type I or Type II PARP inhibitor. A PARPi used in a conjugate, in some aspects, has an apparent equilibrium binding affinity (K_{D}) of less than 100 nM, less than 75 nM, less than 50 nM, less than 25 nM, less than 20 nM, less than 15 nM, less than 10 nm, or less than 5 nM. A PARPi used in a PARPi conjugate, in some aspects, has a dissociation rate constant (k_{d}) of less than 3.0×10⁻³ s⁻¹, less than 2.5×10⁻³ s⁻¹, less than 2.0×10⁻³ s⁻¹, less than 1.5×10⁻³ s⁻¹, or less than 1.0×10⁻³ s⁻¹.

In some aspects of the invention, the PARPi may be a Type III PARPi that has been modified to lower the equilibrium binding affinity (K_{D}) and/or the dissociation rate constant (k_{d}). For example, veliparib has a dissociation rate constant (k_{d}) of 4.08×10⁻³ s⁻¹. For example, Zandarashvili et al. shows that modifying the side group of veliparib, while retaining the core benzimidazole, forms a PARPi (UKTT15) which has a much lower dissociation rate constant (k_{d}) of 1.85×10⁻³ s⁻¹. Such modified PARPi may be used to form PARPi conjugates.

### III. PARP Inhibitor Conjugates

As described herein, a PARPi can be linked or conjugated to a tumor targeting agent (TTA) that binds to a tumor-associated antigen or receptor, or a tumor-specific antigen or receptor for targeted local delivery of the PARPi to tumors. The disclosed conjugates can include coupling of the PARPi to the TTA, or coupling of a radiolabeled PARPi to a TTA, either directly or indirectly. As used herein, “PARPi conjugate” refers to a targeted PARPi, i.e., a PARPi linked or conjugated to a tumor targeting agent (TTA).

For example, in certain aspects of the invention, a PARPi linked or conjugated to a TTA comprises Formula I:

PARPi(Rd)-L-TTA; (I)
wherein Rd is a radiolabel and PARPi(Rd) is a radiolabeled PARPi,
wherein L is a linker and TTA is a tumor targeting agent, and
wherein the L couples the PARPi(Rd) to the tumor targeting agent.

In other aspects of the invention, a PARPi linked or conjugated to a TTA comprises Formula II:

PARPi-L-TTA; (II)
wherein L is a linker and TTA is a tumor targeting agent, and
wherein the L couples the PARPi to the tumor targeting agent.

### IV. Tumor Targeting Agents

PARPi conjugates of the invention include a tumor targeting agent (TTA), which offers yet unrealized advantages in improving the therapeutic index of current and future PARP inhibitors.

A “tumor targeting agent” as used herein is any agent that binds to a target molecule on tumor cells, including a target molecule overexpressed in tumor cells (e.g., expressed to a measurably increased level in tumor cells as compared to non-tumor cells) and/or a target molecule specifically expressed in tumor cells (e.g., substantially not expressed in normal cells). Thus, a tumor targeting agent may bind to a tumor-associated antigen or receptor and/or to a tumor-specific antigen or receptor. A “tumor-associated antigen or receptor” is an antigen or receptor that is found at elevated levels in tumor cells, but that may also be expressed at lower levels in non-tumor cells. A “tumor-specific antigen or receptor” is an antigen or receptor that is only found, or mostly found, in cancer cells. Numerous tumor targeting agents, tumor-associated antigens and receptors, and tumor-specific antigens and receptors are known in the art and routinely used.

A tumor targeting agent that “specifically binds” or “preferentially binds” to a tumor or to a cancer cell is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A TTA “specifically binds” or “preferentially binds” to a target or antigen if it binds with greater affinity, avidity, more readily, and/or with greater duration than it binds to other biomolecules. It is also understood that a TTA that specifically or preferentially binds to a first target may or may not specifically or preferentially bind to a second target. As such, “specific binding” or “preferential binding” does not necessarily require (although it can include) exclusive binding.

Preferably, TTAs used in the invention have high binding affinity, for example, having a dissociation constant K_{D} (k_{off}/kₒₙ) of about 10⁻⁹ M or less.

Representative tumor targeting agents include antibodies, proteins, peptides, aptamers, small molecule inhibitors of a tumor-specific antigen or receptor, small molecule inhibitors of a tumor-associated antigen or receptor (e.g., PMSA inhibitors), and nanoparticles, i.e., any binding agent that shows association with or specific binding to cancer cells.

“Antibodies” are immunoglobulin molecules that recognize and bind to a specific target or antigen, such as a carbohydrate, polynucleotide, lipid, polypeptide, etc., through at least one antigen recognition site, located in the variable region of the immunoglobulin molecule. As used herein, the terms “antibody” and “antibodies” encompass any type of antibody, including but not limited to monoclonal antibodies, polyclonal antibodies, antigen-binding fragments of intact antibodies (e.g., Fab, Fab′, F(ab′)₂, Fd, Fv, Fc, etc.) that retain the ability to specifically bind to a given antigen, bispecific and multispecific antibodies, heteroconjugate antibodies, fusion proteins having an antibody or antigen-binding fragment thereof, (e.g., a domain antibody), single chain antibodies, (scFv), single domain antibody fragments (sdAbs, also known as nanobodies and VHH antibodies), maxibodies, minibodies, intrabodies, diabodies, triabodies, tetrabodies, v-NAR, humanized antibodies, chimeric antibodies, and any other modified configuration of the immunoglobulin molecule that includes an antigen recognition site of the required specificity, including glycosylation variants of antibodies, amino acid sequence variants of antibodies, and covalently modified antibodies.

In certain aspects, antibodies useful in the invention are fully human antibodies, humanized antibodies, or chimeric antibodies.

In other aspects, antibodies useful in the invention are obtained from camelid species such as dromedary camels, Bactrian camels, wild Bactrian camels, llamas, alpacas, vicuñas, and guanacos. In particular, antibodies produced in camelid species have only three hypervariable regions (CDRs), compared to antibody formats derived from conventional IgGs, and as such, these binders preferably recognize and bind conformational epitopes, such as those formed by enzymatic pockets of regulatory domains.

As used herein, the term “antibody” or “antibodies” encompasses antibody mimetics, i.e., having synthetic proteins that show high affinity specific binding similar to antibodies, for example, DARPins, affibodies, knottins, affilins, affimers, affitins, alphabodies, anticalins, avimers, fynomers, kunitx domain peptides, monobodies, nanoCLAMPs, etc.

Representative tumor antigens/receptors to which TTAs useful in the invention bind include, for example, A33, αvβ3, AFP, AKAP-4, ALK, AR, B7-DC (PD-L2), B7H3, B7-H3, BCMA, BCR-ABL, BRCA mutation, BORIS, C1orf186, CA9, CA-125, CA19-9, CA6, CAMPATH-1, CEA, CD19, CD20, CD25, CD30, CD33, CD37, CD45, CD5, CLDN16, CLDN6, CLDN18.2, CMET, CS-1, CCNB1, CYP1B1, DLL3, EGF, EGFR, EGFRVIII, (de2-7 EGFR), EMR², ENG, EPCAM, EPHA2, ERG, ETV6-AML, EWSR¹, FAP, FBP, folate receptor, FOSL1, FRA, FucGM1, G250, GAGE, GD2, GD3, GloboH, GLP-3, GM2, gp100, GRP94 (Endoplasmin), HER², Her-2/neu, HER³, HLA-DR, HMWMAA, HPV E6, HPV E7, hTERT, IL-2 receptor, LCK, LGMN, LewisY, LIV1, LMP2, LRRC₁₅, LY6E, MAD-CT-1, MAD-CT-2, MAGE A1, MAGE A4, MAGE C₂, MAGE-A3, MelanA/MART1, MSLN, ML-IAP, MMPs, MUC1, MUC15, MUC₁₆, MYCN, NA17, NAPI2B, NY-BR-1, NY-ESO-1, OY-TES¹, p53 mutant, p53 nonmutant, PAGE4, PAP, PARPs (e.g., PARP-1), PAX3, PAX5, PDGFR-B, PD-L, PD-L1, PLAV1, polySia, PR¹, PSA, PSCA, PSMA, PTK7, RAS mutations, RGS5, RhoC, RON, ROR1, ROR², SART3, sLe(animal), somatostatin receptor, SP17, SSX2, STn, STRA6, tenascin, TEM1, Tie 3, TIM-3, TMEM238, TMPRSS3, TMPRSS4, RAIL1, TROP2, TRP-2, UPK1B, VEGFR1, VEGFR2, VISTA, VTCN1 (B7-H4), WT1, XAGE 1, and TAG-72. In certain aspects, the TTA binds to a PARP that is also the target of inhibition, e.g., PARP-1.

In a preferred aspect of the invention, the tumor targeting agent is a single domain antibody fragment (sdAb) also known as a VHH molecule or nanobody.

In some aspects of the invention, the tumor targeting agent is “internalizing,” i.e., it is taken up by the cell, along with a PARPi bound to the TTA, upon binding to the target antigen or receptor. As understand in the art, antibodies may be engineered to be internalizing, or otherwise be selected for this property. See e.g., Zhou et al., Arch Biochem Biophys, 2012, 15; 526(2): 107-13.

If the TTA is not naturally internalizing, one or more cell penetration agents may be coupled to the PARPi conjugate and specifically to the TTA to promote intracellular delivery of a PARPi conjugate. Cell penetration agents can protect PARPi conjugate from endosomal entrapment and/or lysosomal degradation.

Representative cell penetration agents include cell-penetrating peptides (CPPs) that are typically 10 to 30 amino acid (aa) peptides in length and are either arginine-rich and amphipathic, or lysine-rich and hydrophobic. A CPP can be, for example, a cationic peptide, amphipathic peptide or hydrophobic peptide, e.g. consisting primarily of Tyr, Trp and Phe, dendrimer peptide, constrained peptide or cross-linked peptide. See e.g., Herce et al, Nat Chem, 2017, 9:762-771.

Following cellular entry, PARPi conjugates of the invention further translocate to the nucleus and bind to DNA. In the context of radiolabeled PARPi conjugates, described elsewhere herein, the PARPi moiety of the conjugate is sufficient for nuclear localization of the radionuclide bound thereto.

In some aspects of the invention, a nuclear localization peptide (NLP) may be additionally used to promote nuclear localization of the PARPi conjugate. For example, karyophilic peptides, composed of at least four arginines, (R), and lysines, (K), within a hexapeptide flanked by proline and glycine helix-breakers, can be used as NLPs for conjugates such as PARPi conjugates. See e.g., Chen et al, J Nucl Med, 2006, 47: 827-836.

### V. Linkers

“Linker (L)” describes the molecule that directly or indirectly links the PARPi to the TTA. A direct linkage of the PARPi to the TTA may be accomplished, e.g., through a direct bond between a functional group of the PARPi and a functional group of the TTA. Alternatively, a linker group may be interposed between the PARPi and the TTA to form an indirect linkage. Indirect attachment of a linker to TTA can be accomplished in a variety of ways, such as, for example with antibodies, through surface lysines, reductive-coupling to oxidized carbohydrates, and through cysteine residues liberated by reducing interchain disulfide linkages or recombinantly added to the protein sequence. A variety of linkage systems are known in the art, including hydrazone-, disulfide-and peptide-based linkages.

In certain aspects of the invention, the linker molecule may be stable (non-cleavable) or cleavable, whereby it is released following cellular entry. In preferred aspects of the invention, the linker is cleavable such that the PARPi is released from the TTA and traffics to the nucleus.

Representative mechanisms by which the PARPi is cleaved from the TTA include hydrolysis at the acidic pH of the lysosomes (hydrazones, acetals, and cis-aconitate-like amides), peptide cleavage by lysosomal enzymes (the cathepsins and other lysosomal enzymes), and reduction of disulfides. As a result of these varying mechanisms for cleavage, mechanisms of linking the PARPi to the TTA also vary widely and any suitable linker can be used.

An example of a suitable conjugation procedure relies on the conjugation of hydrazides and other nucleophiles to the aldehydes generated by oxidation of the carbohydrates that naturally occur on antibodies. Hydrazone-containing conjugates can be made with introduced carbonyl groups that provide the desired drug-release properties. Conjugates can also be made with a linker that has a disulfide at one end, an alkyl chain in the middle, and a hydrazine derivative at the other end.

Linkers containing functional groups other than hydrazones have the potential to be cleaved in the acidic milieu of the lysosomes. For example, conjugates can be made from thiol-reactive linkers that contain a site other than a hydrazone that is cleavable intracellularly, such as esters, amides, and acetals/ketals. Ketals made from a 5 to 7-member ring ketone and that has one of the oxygens attached to the cytotoxic agent and the other to a linker for TTA attachment also can be used.

Another example of a class of pH sensitive linkers are the cis-aconitates, which have a carboxylic acid juxtaposed to an amide bond. The carboxylic acid accelerates amide hydrolysis in the acidic lysosomes. Linkers that achieve a similar type of hydrolysis rate acceleration with several other types of structures can also be used.

Another potential release method for releasing PARPi from the conjugate is the enzymatic hydrolysis of peptides by the lysosomal enzymes. In one example, a peptide is attached via an amide bond to para-aminobenzyl alcohol and then a carbamate or carbonate is made between the benzyl alcohol and the cytotoxic agent. Cleavage of the peptide leads to the collapse, or self-immolation, of the aminobenzyl carbamate or carbonate. In one example, a phenol can also be released by collapse of the linker instead of the carbamate. In another variation, disulfide reduction is used to initiate the collapse of a para-mercaptobenzyl carbamate or carbonate.

Exemplary cleavable linkers are taught in the following references, each of which are incorporated herein by reference: Bargh et al. Chem Soc Rev, 2019, 48:4361-4374, Leriche et al., Bioorg Med Chem, 2012, 20:571-582, Bohme et al. J Pept Sci, 2015, 21:186-200, and Kern et al. Bioconjug Chem, 2016, 27:2081-2088.

Site-specific conjugation of antibodies and proteins mediated by a transglutaminase has been described previously, PCT International Publication No. WO 2012059882, which is incorporated herein by reference. It was described that an Fc-containing polypeptide engineered with an acyl donor tag (e.g., Gln containing peptide tags), in the presence of transglutaminase, can covalently crosslink with an amine donor agent (e.g., a small molecule comprising or attached to an amine donor unit) to form a homogenous population of an engineered Fc-containing polypeptide conjugate with the amine donor agent being site-specifically conjugated to the Fc-containing polypeptide through the acyl donor tag. The conjugation efficiency of the Fc-containing polypeptide engineered with an acyl donor tag and the amine donor agent is at least about 51%, and the conjugation efficiency between the Fc-containing polypeptide and the amine donor agent is less than about 5% in the absence of the acyl donor tag. Further, deletion or mutation of the last amino acid from Lys (lysine) to another amino acid in the Fc-containing polypeptide spatially adjacent to the Gln-containing peptide tag provides a significant increase in conjugation efficiency of the Fc-containing polypeptides and the small molecule. Further, the selection of the acyl donor tags, Fc-containing polypeptides, and/or the amine donor agents as described allows for site-specific conjugation. This strategy can be applied to the formation of a PARPi conjugate by coupling a PARPi to a TTA.

Some PARPi conjugates may have limited water solubility which can contribute to aggregation of the conjugate. One approach to overcoming this is to add hydrophilic groups to the linker. An example of a hydrophilic group used in the linker is polyethylene glycol (PEG). In one aspect, the hydrophilic group may be linked to a cleavable linker to form a hydrophilic/cleavable linker. In one specific example, the hydrophilic group is PEG and the cleavable linker is a dipeptide that is cleaved by lysosomal enzymes.

In one aspect of a hydrophilic linker, the hydrophilic group (e.g., PEG) will have coupling groups on each end which are used to form bonds with the PARPi component and a dipeptide cleavable linker. The cleavable linker, in this aspect, is coupled to the TTA. Exemplary end groups include, but are not limited to carboxyls, hydroxyls, thiols, and amines. End groups may be the same, or may be different. Such groups can form bonds with complementary groups on the PARPi, at one end, and the cleavable linker at the other end.

In another aspect, the hydrophilic group (e.g., PEG) may be positioned between the cleavable linker and the TTA. The end group at one end of the hydrophilic group is selected for coupling to the cleavable linker (e.g., a dipeptide) and can include groups such as carboxyls, hydroxyls, thiols, and amines. The other end of the hydrophilic group can have a group for coupling to the TTA. When the TTA is a polypeptide (e.g., an antibody) one end group of the hydrophilic group may be a maleimide group. The maleimide group commonly forms bonds to polypeptides through thiol groups (e.g., on cysteine amino acids). Other thiol-reactive groups include haloacetyls, aziridines, acryloyls, arylating agents, vinylsulfones, and pyridyl disulfides.

In some aspects of the invention, the linker component of the PARPi conjugates of the present invention have the formula:

ES¹-SS-ES²
wherein ES¹ has the structure: Cg¹-SP-;
wherein SS is a disulfide bond;
wherein ES² has the structure: -SP-Cg²-;
wherein Cg¹ and Cg² are each, independently, succinimidyloxycarbonyl, maleimide, —OC(O)—;
—C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N—C—; and —C═N—N(R¹)—;
wherein R¹ and R² are independently selected from H, branched or unbranched C₁₋₁₀ alkyl, or aryl;
wherein SP is —C₁-C₁₀ alkylene-; —C₁-C₁₀ alkenylene-; —C₁-C₁₀ alkynylene-; carbocyclo-, —O—(C₁-C₈ alkylene)-; O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣCH₂—,
   wherein r is an integer ranging from 1-10;
wherein Cg³ is a coupling group that forms a covalent bond to the Rd; and wherein Cg⁴ is a coupling group that forms a covalent bond to the TTA.

A linker comprising a disulfide linker is cleavable under reducing conditions. A variety of disulfide linkers are known in the art, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyloxycarbonyl-alpha-methyl-alpha-(2-pyridyl-dithio)toluene). Disulfide linkers are described in Thorpe et al., Cancer Res., 1987, 47:5924-5931; Wawrzynczak et al., In Immunoconjugates: Antibody Conjugates in Radioimaging and Therapy of Cancer (C. W. Vogel ed., Oxford U. Press, 1987 and U.S. Pat. No. 4,880,935, each of which is incorporated herein by reference in its entirety.

In some aspects of the invention, the linker component of the disclosed PARPi conjugate has the formula:

ES-ECL-SIG
wherein ES has the structure: Cg³-SP-Cg⁴;
Cg³ and Cg⁴ are each, independently, succinimidyloxycarbonyl; maleimide; —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or Cg³ and Cg⁴ are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl; and wherein Cg³ is a coupling group that forms a covalent bond to the Rd component and Cg⁴ is a coupling group that forms a covalent bond to ECL;
SP is —C₁-C₁₀ alkyl; —C₁-C₁₀ alkylene; —(CH₂CH₂O)ᵣ—; —CH₂—(CH₂CH₂O)ᵣ—; —(CH₂CH₂O)ᵣ—CH₂—; or —CH₂—(CH₂CH₂O)ᵣ—CH₂—;
ECL is an enzyme cleavable linker; and
SIG is a self-immolative group that forms a covalent bond between the ECL and the TTA, wherein upon cleavage of the ECL, the SIG decomposes to release the TTA component from the ECL component.

An enzyme cleavable linker (ECL) is a linker that is specifically cleaved by an enzyme. In one embodiment, an ECL is cleaved by a lysosomal enzyme. Particularly useful lysosomal enzymes include lysosomal glycosidases and lysosomal proteases. Specific examples of lysosomal enzymes include, but are not limited to cathepsin proteases (e.g., Cathepsin B) and glycosidases (e.g., β-glucuronidase and β-galactosidase).

The ECL linker, in one embodiment, is a cleavable peptide linker having between two to four amino acids. Exemplary dipeptide linkers that are cleavable by a lysosomal enzyme include, but are not limited by, Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; Trp-Cit.

In other embodiments, the ECL comprises a glucuronide unit that can be cleaved by a lysosomal glycosidase enzyme (See, for example, US 2012/0107332, incorporated by reference herein). In some embodiments, the glucuronide unit of an ECL comprises a sugar moiety (Su) linked via a glycoside bond (—O′—) to a self-immolative group (Z) of the formula as depicted below (See also US 2012/0107332, incorporated by reference herein).

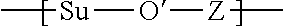

The glycosidic bond (—O′—) is typically a β-glucuronidase-cleavage site, such as a bond cleavable by human, lysosomal β-glucuronidase. In the context of a glucuronide unit, the term “self-immolative group” (SIG) refers to a di-or tri-functional chemical moiety that is capable of covalently linking together two or three spaced chemical moieties (i.e., the sugar moiety (via a glycosidic bond), a PARPi (directly or indirectly via a spacer unit), and, in some embodiments, a linker (directly or indirectly via a stretcher unit) into a stable molecule. The self-immolative group, in this embodiment, will spontaneously separate from the first chemical moiety (e.g., the spacer or PARPi unit) if its bond to the sugar moiety is cleaved. p-Aminobenzyl alcohol (PABA) is an example of a SIG that is commonly used.

In some embodiments, the sugar moiety (Su) is a cyclic hexose, such as a pyranose, or a cyclic pentose, such as a furanose. In some embodiments, the pyranose is a glucuronide or a hexose. The sugar moiety is usually in the β-D conformation. In a specific embodiment, the pyranose is a β-D-glucuronide moiety (i.e., β-D-glucuronic acid linked to the self-immolative group —Z— via a glycosidic bond that is cleavable by β-glucuronidase). In some embodiments, the sugar moiety is unsubstituted (e.g., a naturally occurring cyclic hexose or cyclic pentose). In other embodiments, the sugar moiety can be a substituted β-D-glucuronide (i.e., glucuronic acid substituted with one or more group, such hydrogen, hydroxyl, halogen, sulfur, nitrogen or lower alkyl. Additional exemplary glucuronide units are described in US. Patent Application Publication No. 2012/0107332, which is incorporated herein by reference.

The linker, in some embodiments, includes a spacer (SP) that links one or more components of the PARPi conjugate. The spacer can serve a number of purposes, depending on the location of the spacer in the PARPi conjugate and the composition of the spacer. A spacer may allow an enzyme-cleavable linker or a disulfide linker to be transformed and/or cleaved with more ease. A spacer may be used to improve the pharmacokinetic properties, solubility, or aggregation behavior of the PARPi conjugate. Use of a spacer may make the PARPi more accessible to the PARP (e.g., PARP-1), by separating the PARPi from the TTA. A spacer may also provide for better accessibility to the linker, which, especially in the case of enzymatic cleavage or transformation may improve the rate at which the linker is transformed and/or cleaved. The spacer may be a water-soluble moiety or contain one or more water-soluble moieties, such that the spacer contributes to the water solubility of the PARPi conjugate.

In one embodiment, a spacer is selected from —C₁-C₁₀ alkylene-; —C₁-C₁₀ alkenylene-; —C₁-C₁₀ alkynylene-; carbocyclo-; —O—(C₁-C₈ alkylene)-; O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; —C₂-C₁₀ alkynylene-(carbocyclo)-; -(carbocyclo)-C₁-C₁₀ alkylen-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—; and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted.

Other representative linkers contain two functional groups, including (1) a group (e.g., carboxylic acid) for coupling with a TTA, and (2) a carbonyl group (e.g., an aldehyde or a ketone) for coupling with a PARPi. The carbonyl group will react with a hydrazide group on the PARPi to form a hydrazone linkage. This linkage is cleavable by hydrolysis, allowing for release of the PARPi from the conjugate after binding to the target. See e.g., U.S. Pat. No. 5,773,001, which is incorporated herein by reference. In some aspects of the invention, the hydrolyzable linker used is 4-(4-acetylphenoxy) butanoic acid (AcBut). In other aspects of the invention, the linkage can be prepared using (3-Acetylphenyl) acetic acid (AcPAc) or 4-mercapto-4-methyl-pentanoic acid (Amide) as the linker molecule. Such a linker is cleavable under intracellular conditions such that cleavage of the linker sufficiently releases the PARPi from the TTA into the intracellular environment, but is inhibited from releasing the PARPi into the extracellular environment. In some embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. See, e.g., U.S. Pat. Nos. 5,122,368; 5,824,805; 5,622,929; Dubowchik and Walker, 1999, Pharm. Therapeutics 83:67-123; Neville et al., 1989, Biol Chem 264:14653-14661, all of which are incorporated herein by reference. These linkers are relatively stable under neutral pH conditions, such as those in the blood, but are unstable between pH 4.5 to 5.0, the approximate pH of the lysosome. In certain embodiments, the cleavable linker is a thioether linker {such as, e.g., a thioether attached to the therapeutic agent via an acylhydrazone bond (see, e.g., U.S. Pat. No. 5,622,929, which is incorporated herein by reference).

Other linkers include groups such as N-hydroxysuccinimide (NHS) esters, sulfo-NHS (sulfonated NHS) esters, PFP (pentafluorophenyl) esters, TFP (tetrafluorophenyl) esters, 4-nitrophenyl esters and DNP (dinitrophenyl) esters.

The PARPi component of the disclosed PARPi conjugates may be linked to the TTA using a carbodiimide reagent. For example, a PARPi, optionally radiolabeled with a radionuclide, can be conjugated to a TTA using a carbodiimide reagent which readily reacts with nitrogen or oxygen containing side chains of the TTA. In one embodiment, the PARPi is coupled to a linker using a carbodiimide reagent. Examples of carbodiimides that can be used include, but are not limited to, 1-ethyl-3-(3-dimethylaminopropyl)carbodiimide (EDC); N,N′-dicyclohexyl carbodiimide (DCC); N,N′-diisopropyl carbodiimide (DIC); N-cylcohexyl-N′-(2-morpholinoethyl) carbodiimide; N-cylcohexyl-N′-[2-(4-methylmorpholin-4-ium-4-yl)ethyl] carbodiimide tosylate; N-cylcohexyl-N′-[4-(diethylmethylammonio)cyclohexyl] carbodiimide tosylate; N,N′-bis(2,2-dimethyl-1,3-dioxolan-4-yl)methyl]carbodiimide; and N-benzyl-N′-isopropylcarbodiimide.

In one aspect, the linker has a functionality that is capable of reacting with a free cysteine present on a TTA to form a covalent bond. Nonlimiting examples of such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., Klussman, et al, Bioconjugate Chemistry, 2004, 15(4):765-773 and Abad et al. Chem. Commun., 2012, 48, 6118-6120, which are both incorporated herein by reference.

In some embodiments, the linker has a functionality that is capable of reacting with an electrophilic group present on a TTA. Exemplary electrophilic groups include, but are not limited to, aldehyde and ketone carbonyl groups. In some embodiments, a heteroatom of the reactive functionality of the linker can react with an electrophilic group on a TTA and form a covalent bond to the TTA. Nonlimiting examples of such reactive functionalities include, but are not limited to, hydrazide, oxime, amino, hydrazine, thiosemicarbzide, hydrazine carboxylate, and aryl hydrazide.

Functional groups that can be used to link the TTA component to the PARPi component, either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an antibody. In another embodiment, sulfhydryl groups can be generated by reaction of an amino group of the TTA with 2-iminothiolane (Traut's reagent) or other sulfhydryl generating reagents. In certain embodiments, the TTA is a recombinant antibody and is engineered to carry one or more additional amino containing groups (e.g., lysine). In certain other embodiments, the TTA is a recombinant antibody that is engineered to carry additional sulfhydryl groups, e.g., additional cysteines.

In other aspects of the invention, the linker may couple the TTA to the PARPi component through a dipeptide linker, such as a valine-citrulline (Val-Cit), a phenylalanine-lysine (Phe-Lys) linker, or maleimidocaproyl-valine-citrulline-p-aminobenzyloxycarbonyl (vc) linker. In another aspect, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Further, the linker may be maleimidocaproyl (mc).

In a preferred embodiment, the TTA is conjugated to a PARPi inhibitor (optionally radiolabeled), via a linker comprising maleimidocaproyl (“mc”), valine-citrulline (Val-Cit or “vc”), and PABA (referred to as a “mc-vc-PABA linker”). Maleimidocaproyl acts as a linker to the TTA and is not cleavable. Val-Cit is a dipeptide linker, which is cleavable by a lysosomal protease, specifically the protease cathepsin B. Thus, the Val-Cit component of the linker provides a means for releasing the PARPi from the PARPi conjugate upon exposure to the intracellular environment. Within the linker, p-aminobenzylalcohol (PABA) acts as a spacer and is self immolative, allowing for the release of the PARPi.

### VI. Radiolabeled and Photsensitizing PARP Inhibitor Conjugates

With respect to therapy, because PARP-1 is a nuclear enzyme, use of radionuclides emitting short-range radiation are most appropriate. Both Auger electrons and the recoil nuclei of alpha particles belong to this category. Because the DNA is the most sensitive part of the cell to radiation, PARP-1 targeting of the radionuclide is very helpful in enhancing efficacy. Also, because these radiations are of short range, destruction of neighboring normal tissues can be minimized. Shifting the site of radioactive decay from other parts of the cell to the cell nucleus increases the geometrical probability (solid angle) that the path of the radioactive particle will pass through the cell nucleus and thus increases therapeutic effectiveness.

“Radionuclide” or “radionuclides”, as used herein, refers to an unstable form of a chemical element that releases radiation as it breaks down and becomes more stable. This excess energy can be released by emission of gamma radiation; emission of an Auger electron, emission of a beta particle, emission of conversion electrons, or emission of an alpha particle. Auger electrons and alpha particles are particularly useful for the treatment of cancer. Auger electrons, alpha particles, and the recoil nucleus created during alpha-particle emission have a short range and high linear energy transfer making them effective agents for selective targeting and treatment of cancer cells.

Preferably, the radionuclide is bound directly, or coupled via radiolabeled prosthetic agents, to the PARPi. Radionuclides suitable for radiotherapy include but are not limited to α-emitters, β-emitters, and Auger electron emitters.

Examples of radionuclides include, but are not limited to: ³H, ¹¹C, ¹³N, ¹⁴C, ¹⁵N, ¹⁵O, ³⁵S, ¹⁸F, ³²P, ³³P, ⁴⁷Sc, ⁵¹Cr, ⁵⁷Co, ⁵⁸Co, ⁵⁹Fe, ⁶²Cu, ⁶⁴Cu, ⁶⁷Cu, ⁶⁷Ga, ⁶⁸Ga, ⁷⁵Se, ⁷⁶Br, ⁷⁷Br, ⁸⁶Y, ⁸⁹Zr, ⁹⁰Y, ⁹⁴Tc, ⁹⁵Ru, ⁹⁷Ru, ⁹⁹Tc, ¹⁰³Ru, ¹⁰⁵Rh, ¹⁰⁵Ru, ¹⁰⁷Hg, ¹⁰⁹Pd, ¹¹¹Ag, ¹¹¹ In, ¹¹³ In, ¹¹⁹Sb, ¹²¹Te, ¹²²Te, ¹²³I, ¹²⁴I, ¹²⁵I, ¹²⁵Te, ¹²⁶I, ¹³¹I, ¹³¹ In, ¹³³I, ¹⁴²Pr, ¹⁴³Pr, ¹⁵³Pb, ¹⁵³Sm, ¹⁶¹Tb, ¹⁶⁵Tm, ¹⁶⁶Dy, ¹⁶⁶H, ¹⁶¹Tb, ¹⁶⁷Tm, ¹⁶⁸Tm, ¹⁶⁹Yb, ¹⁷⁷Lu, ¹⁸⁶Re, ¹⁸⁸Re, ¹⁸⁹Re, ¹⁹¹Pt, ^{193m}Pt, ^{195m}Pt, ¹⁹⁷Pt, ¹⁹⁷Hg, ¹⁹⁸Au, ¹⁹⁹Au, ²⁰¹Tl, ²⁰³Hg, ²¹¹At, ²¹²Bi, ²¹²Pb, ²¹³Bi, ²²³Ra, ²²⁴Ra, ²²⁴Ac, ²²⁵Ac, and ²²⁷Th.

In preferred aspects of the invention, PARPi conjugates comprise short range emitting radionuclides such as ¹²³I, ¹²⁵I, ⁷⁷Br, and ²¹¹At.

Representative methods for radiolabeling PARP inhibitors and PARPi conjugates of the invention are disclosed in US Published Application No. US20200188541, which is incorporated by reference herein in its entirety. Radiolabeling of PARP inhibitors may necessitate modification of the chemical structure of the PARP inhibitor to allow coupling of a radionuclide to the inhibitor. The modified PARP inhibitor will retain the portions of the molecule necessary for nuclear localization, binding to PARP and subsequent DNA binding, and may or may not retain portions of the molecule necessary for inhibitory effects. Some modifications to a PARPi may be made without changing the ability of the PARPi to bind and inhibit PARP. Other modifications to a PARPi, may not affect the ability of the PARPi to bind to PARP, but the modification may change the ability of the modified PARPi to inhibit PARP.

In one aspect of the invention, a radionuclide component is coupled to a PARPi conjugate having the structure:

PARPi(Rd)-linker-TTA;
wherein Rd is a component of the PARPi conjugate that comprises a radionuclide and is coupled to the PARPi; TTA is a tumor targeting agent; and linker couples the PARPi(Rd) to the TTA.

In one aspect of the invention the Rd component has the structure:

Cg¹-ArQ-Cg²;
wherein Cg¹ is a coupling group that couples ArQ to the PARPi, and Cg² is a coupling group that couples Rd to the linker;
wherein Cg¹ and Cg² are each, independently, succinimidyloxycarbonyl; maleimide;
—OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N—C—; —N(R¹)—N═; ═N—N(R¹)—; —N(R¹)—N═C—; or —C═N—N(R¹)—; or
   Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N—; ═N—O—; —C═N—O—; —O—N═C—; —N(R¹)—N═; ═N—N(R¹)—; —N(R¹)—N═C—; and —C═N—N(R¹)—;
   wherein R¹ and R² are independently selected from H, branched or unbranched C₁₋₁₀ alkyl, or aryl;
wherein Ar is an aryl, a heteroaryl, or a radionuclide chelating agent; and
wherein Q is a radionuclide.

In one aspect of the invention, the linker in a radiolabeled PARPi conjugate has the structure:

ES¹-SS-ES²
wherein ES¹ has the structure: Cg³-SP-;
wherein SS is a disulfide bond;
wherein ES² has the structure: -SP-Cg⁴-;
wherein Cg³ and Cg⁴ are each, independently, succinimidyloxycarbonyl; maleimide;
—OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C—N—O—; —O—N—C—; —N(R¹)—N═; ═N—N(R¹)—; —N(R¹)—N═C—; or —C═N—N(R¹)—; or Cg³ and Cg⁴ are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N—C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein SP is —C₁-C₁₀ alkylene-; —C₁-C₁₀ alkenylene-; —C₁-C₁₀ alkynylene-; carbocyclo-; —O—(C₁-C₈ alkylene)-; O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—,
   wherein r is an integer ranging from 1-10;
wherein Cg³ is a coupling group that forms a covalent bond to the Rd; and wherein Cg⁴ is a coupling group that forms a covalent bond to the TTA.

In another aspect of the invention, the linker in a radiolabeled PARPi conjugate has the structure:

ES-ECL-SIG
wherein ES has the structure: Cg³-SP-Cg⁴;
Cg³ and Cg⁴ are each, independently, succinimidyloxycarbonyl, maleimide, —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; or —C═N—N(R¹)—; or Cg³ and Cg⁴ are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N—C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein Cg³ is a coupling group that forms a covalent bond to the Rd component and Cg⁴ is a coupling group that forms a covalent bond to ECL;
wherein SP is —C₁-C₁₀ alkylene-; —C₁-C₁₀ alkenylene-; —C₁-C₁₀ alkynylene-; carbocyclo-; —O—(C₁-C₈ alkylene)-; —O—(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—, wherein r is an integer ranging from 1-10;
wherein ECL is an enzyme cleavable linker; and
wherein SIG is a self-immolative group that forms a covalent bond between the ECL and the TTA, wherein upon cleavage of the CL, the SIG decomposes to release the TTA from the ECL.

Aryl groups include, but are not limited to, benzene, naphthalene, anthracene, acenaphthylene, anthracenone, anthracenedione, biphenylene, fluorene, fluoren-9-one, phenanthrene, phenanthrenedione, pyrene, and triphenylene; aryl groups may be unsubstituted or substituted with one or more of cyano, nitro, hydroxyl, alkoxyl; mercapto, amino, carboxyl, aminocarbonyl, aminothiocarbonyl, sulfo, aminosulfonyl, halogen, and C₁-C₆-alkyl.

Aromatic heterocyles include, but are not limited to, acridine, benzocinnoline, benzofuran, benzoquinoline, benzothiophene, benzopyran, carbazole, cinnoline, dibenzothiophene, furan, imidazole, indazole, indole, indolizine, isobenzofuran, isoindole, isoquinoline, isothiazole, naphthyridine, oxazole, phenanthrothiophene, phenanthridine, phenanthroline, phenazine, phthalazine, pteridine, purine, pyrazine, pyrazole, pyridazine, pyridine, pyrimidine, pyrrole, quinazoline, quinoline, quinoxaline, tetrazole, thiazole, thiophene, triazine, and xanthene.

In some embodiments, a radionuclide is bound to a chelating agent. The chelating agent is coupled to the PARPi. Representative radionuclide chelating agents useful for complexation of radionuclides include tetraxetan (DOTA), 1,4,7-triazacyclononane-1,4,7-triacetic acid (NOTA), diethylenetriamine pentaacetate (DTPA)-isothiocyanate, succinimidyl 6-hydrazinium nicotinate hydrochloride (SHNH), and hexamethylpropylene amine oxime (HMPAO) (See Bakker et al, J Nucl Med, 1990, 31: 1501-1509, Chattopadhyay et al, Nucl Med Biol, 2001, 28: 741-744; Dewanjee et al, J Nucl Med, 1994 35: 1054-63; Krenning et al, Lancet, 1989, 1: 242-244; Sagiuchi et al, Ann Nucl Med, 2001, 15: 267-270); U.S. Pat. No. 6,024,938, all of which are incorporated herein by reference). Radiohalogenation methods are also known in the art, and representative protocols may be found, for example, in Krenning et al, Lancet, 1989, 1:242-4; Bakker et al, J Nucl Med, 1990, 31:1501-9; Coenen et al., Radiochimica Acta 1983; 34: 47-68 and Lewis et al (Eds) Radiopharmaceutical Chemistry, Springer 2019, all of which are incorporated herein by reference.

In some aspects, including the above described embodiment, the ECL is optionally cleavable by a lysosomal enzyme, such as a lysosomal protease or a lysosomal glycosidase, for example a peptide composed of two to four amino acids.

In other aspects of the invention, PARPi conjugates comprise a photosensitizing agent such as chlorin(e6), porphyrins, or IRDye700DX in place of the radiolabel Rd as described above.

### VII. Therapeutic Uses

It will be appreciated that the PARPi conjugates of the instant invention may be used for treatment of cancer or other neoplastic disorders, whether administered alone or in combination with an additional anti-cancer agent or radiotherapy. The disclosed PARPi conjugates are particularly useful for generally treating neoplastic conditions in subjects with benign or malignant solid tumors.

### A. Radiosensitization Using PARP Inhibitor Conjugates

PARPi conjugates of the invention may be used to radiosensitize tumors to thereby allow lower levels of ionizing radiation (IR), preferably otherwise non-toxic doses, to be therapeutically effective. Without wishing to be bound by any theory, the sensitivity may be attributable to a DNA replication process during which replication forks collapse during delayed single strand break repair, for example, as described in Dungey et al, Int J Radiat Oncol Biol Phys, 2008, 72(4): 1188, which is incorporated herein by reference. Alternatively, the sensitivity may be attributable to DNA double strand breaks generated by collapse of stalled replication forks during DNA replication. When a subject is to be treated with IR, an effective dose of the PARPi conjugate for radiosensitization may be less than a cytotoxic dose of the same PARPi conjugate, i.e., a dose that is therapeutically effective as a single agent. In certain aspects of the invention, radiosensitization using the disclosed PARPi conjugates are quantified in terms of an enhancement ratio, which is defined as the mean effective dose of control to treatment, and is preferably at least 1.2, at least 1.3, at least 1.4, at least 1.5, or more.

### B. Therapy Using Radiolabeled PARP Inhibitor Conjugates

The radiolabeled PARPi conjugates disclosed herein may be used as first in class targeted therapies for the treatment of cancer, particularly those cancers characterized by solid tumors. A significant advantage of the disclosed PARPi conjugates is optimization of the PARPi therapeutic index by reducing or eliminating side effects that occur as a result of nonspecific targeting of a therapeutic radionuclide. As such, the disclosed PARPi conjugates may be used at reduced doses and/or less aggressive administration regimens as a result of the improved therapeutic index. Specifically, PARPi conjugates as disclosed herein may show improved therapeutic outcomes, including but not limited to reduction of tumor size, delayed tumor growth, fewer metastases, and/or increased longevity. Such improvements are at least about 10%, or at least about 20%, or at least about 30%, or at least about 40%, or at least about 50%, or at least about 60%, or at least about 70%, or at least about 80%, or at least about 90%, or at least about 95%, or at least about 99%, when compared to a therapeutic effect observed in a subject receiving the directly labeled PARPi only, i.e., without targeting as in a PARPi conjugate described herein (See Laird et al. J. Thoracic Oncology 2019, 14(10): 1743-1752 and Makvandi et al. Mol. Cancer Ther. 2019, 18(7):1195-1204, both of which are incorporated herein by reference). In other aspects, such improvements are at least about 2-fold, or at least about 5-fold, or at least about 10-fold, or at least about 20-fold, or at least about 50-fold, or at least about 100-fold, or more, when compared to any therapeutic effect observed in a subject receiving the PARPi only, i.e., without targeting as in a PARPi conjugate described herein.

Likewise, the PARPi conjugates disclosed herein are designed to have an improved therapeutic effect as compared to a tumor cell targeted radionuclide that lacks the additional PARPi nuclear targeting component, i.e., a radiolabeled TTA. Specifically, radioactive decay is more toxic in the nucleus as compared to the cytoplasm or cell surface.

The effectiveness of short-range radionuclides can be determined using the MIRD S-value calculations of Goddu et al. MIRD Cellular S-Values, 1997, Reston, VA Society of Nuclear Medicine, which is incorporated herein by reference, to compare the radiation dose that the nucleus of a cell would receive if the site of the radioactive decay was in the nucleus, S(N←N), in the cytoplasm, S(N←Cy), or on the cell surface S(N ←CS). These calculations are based on observations that typical cancer cells have a cell radius of about 9 μm and a nuclear radius of about 7 μm. See Akabani et al. Nucl Med. Biol. 2006, 33:333-347. The S-values for some clinically relevant radionuclides for therapy with different types of therapeutic radiation are summarized in Table 1. Notably, these calculations do not include effects from the alpha decay recoil nucleus, and therefore, are likely underestimated values for the alpha emitters.

The dose enhancement ratio is the gain in radiation dose obtainable by moving the site of decay from the cytoplasm or the cell surface to the cell nucleus and is calculated as:

S(N←N)/S(N←Cy) and S(N←N)/S(N←CS), respectively,

As can be seen from the Table 1, shifting the site of radioactive decay from the cytoplasm to the cell nucleus enhances the radiation dose to the radiation sensitive element of the cell—its nucleus by a factor of 2.3 for alpha particle emitters, by a factor of 2.7-3.0 for beta-particle emitters and a factor of 7.0-8.4 for Auger electron emitters. Likewise, shifting the site of radioactive decay from the cell surface to the cell nucleus enhances the radiation dose to the cell nucleus by a factor of 3.1-3.2 for alpha particle emitters, by a factor of 3.8-4.4 for beta-particle emitters and a factor of 10.6-39.3 for Auger electron emitters.

TABLE 1
Effect of Moving the Site of Radioactive Decay on the Radiation Dose Delivered
to the Cell Nucleus for Radionuclides of Interest for Targeted Radiotherapy

| | | | | | Dose Enhancement | |
|---|---|---|---|---|---|---|
| | | | | | Ratio | |
| | Therapeutic | S value (Gy/Bq s) | | | Nucleus/ | Nucleus/ |
| Radionuclide | Radiation | S(N←N) | S(N←Cy) | S(N←CS) | Cytoplasm | Cell Surface |
| Ga-67 | Auger | 7.43E−04 | 9.32E−05 | 2.84E−05 | 8.0 | 26.2 |
| Br-77 | Auger | 5.23E−04 | 6.52E−05 | 1.33E−05 | 8.0 | 39.3 |
| In-111 | Auger | 6.02E−04 | 8.59E−05 | 5.70E−05 | 7.0 | 10.6 |
| I-123 | Auger | 6.29E−04 | 7.49E−05 | 4.93E−04 | 8.4 | 12.8 |
| I-131 | Beta-particle | 3.18E−04 | 1.19E−04 | 8.47E−05 | 2.7 | 3.8 |
| Lu-177 | Beta-particle | 4.72E−04 | 1.55E−04 | 1.05E−04 | 3.0 | 4.4 |
| At-211 | Alpha-particle | 4.35-E−02 | 1.91E−02 | 1.41E−02 | 2.3 | 3.1 |
| Bi-213 | Alpha-particle | 1.25E−03 | 5.38E−04 | 3.96E−04 | 2.3 | 3.2 |
| Ac-225* | Alpha-particle | 4.88E−02 | 2.16E−02 | 1.60E−02 | 2.3 | 3.1 |

*Not including dose from radioactive daughters or the like.

Thus, the PARPi(Rd) conjugates of the invention may show radiation dose enhancement ratios of at least about 2 or more when compared to a radiolabeled TTA comprising the same TTA and same radionuclide as the PARPi(Rd) conjugate. For example, the dose enhancement ratio may be at least about 3 or more, or 4 or more, or 5 or more, and in some aspects, at least 10 more, such as 20 or more, 25 or more, 30 or more, 40 or more, or 50 or more.

### C. Subjects

The PARPi conjugates disclosed herein may be used to treat any of various cancers and other neoplastic conditions as are recognized in the art. In certain aspects, the PARP conjugates are used to treat cancers associated with solid tumors, including, for example, tumors of the ovary, breast, adrenal, liver, kidney, bladder, breast, gastrointestinal tract, cervix, uterus, prostate, pancreas, lung, thyroid, and brain. In particular, inhibition of PARP-1 has been shown to be effective against many types of tumors. In preferred aspects of the invention, subjects with ovarian, brain or breast cancer are treated using the disclosed PARPi conjugates.

In related aspects of the invention, the disclosed PARPi conjugates and methods may be used to treat tumors, including any of the aforementioned tumors, that are characterized by repair proteins that are deficient or defective, for example, tumors having deficient in at least one gene involved in the homologous recombination repair (HRR) pathway. Representative genes involved in the homologous recombination repair pathway include BRCA1 or BRCA2, as well as numerous other genes, for example as described in US20210106574, herein incorporated by reference in its entirety.

### D. Formulation, Administration, and Dose

The disclosed PARPi conjugates of the invention may be formulated as desired using art-recognized techniques. In some embodiments, the therapeutic compositions of the invention may be administered neat or with a minimum of additional components while others may optionally be formulated to contain suitable pharmaceutically acceptable carriers (e.g., vehicles, adjuvants, and diluents) comprising excipients and auxiliaries that are well known in the art, including for example, pH adjusting and buffering agents, tonicity adjusting agents, stabilizers, wetting agents, radioprotectants and the like. Certain non-limiting exemplary carriers include saline, buffered saline, dextrose, water, glycerol, ethanol, and combinations thereof. Certain non-limiting exemplary radioprotectants include ascorbic acid, gentisic acid, ethanol and combinations thereof.

In general, the compounds and compositions of the invention may be administered to a subject by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms suitable for the particular mode of administration, including, for example, tablets, capsules, powders, granules, ointments, solutions, suppositories, enemas, injections, inhalants, and aerosols.

The particular dosage regimen for administering PARPi conjugates of the invention, i.e., dose, timing and repetition, will depend on the particular subject and that subject's medical history, as well as empirical considerations such as pharmacokinetics (e.g., half-life, clearance rate, etc.). Frequency of administration may be determined and adjusted over the course of therapy. A therapeutically effective dose is a dose sufficient to provide a clinical benefit to the subject, including for example, a dose sufficient to reduce tumor size, maintain a reduction of tumor size, reduce or slow tumor growth, delay the development of metastasis, improve longevity, etc. Dosage administered may be adjusted or attenuated to manage potential side effects and/or toxicity.

### EXAMPLES

The following examples are included to demonstrate certain embodiments of the invention.

### Example 1. Preparation of Radiolabeled Olaparib Synthon With Valine-Citrulline-PABC Linker

Scheme 1 depicts an example of a method of making a synthon (6) that incorporates radiolabeled olaparib as a PARP-1 inhibitor (PARPi) which can be coupled to an antibody that binds to a tumor-associated antigen/receptor or a tumor-specific antigen/receptor. As used herein a “synthon” is a synthetic intermediate that includes the PARPi and a linker. The synthon is coupled to an antibody to form the PARPi conjugate.

Olaparib (1) is a PARP inhibitor that is used for the treatment of BRCA-mutated ovarian cancer. To facilitate coupling of olaparib to an antibody, the cyclopropyl ketone end-group is removed, leaving a piperazine ring as a functional group for attachment to the antibody, olaparib derivative (2).

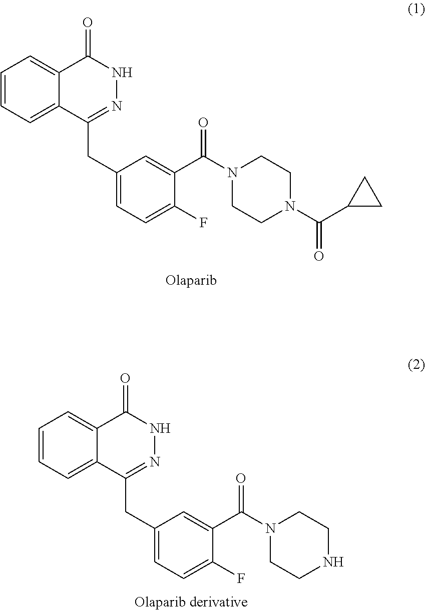 [SMILES: O=C(c1cc(Cc2n[nH]c(=O)c3ccccc23)ccc1F)N1CCN(C(=O)C2CC2)CC1 ; O=C(c1cc(Cc2n[nH]c(=O)c3ccccc23)ccc1F)N1CCNCC1]

As depicted in Scheme 1, an analogue of olaparib (1) is prepared by reacting olaparib derivative (2) with an iodobenzene moiety-containing molecule (3) having two coupling groups (Cg, succinimidyloxycarbonyl) to form the intermediate (4). Radiolabeled intermediate (4) can be synthesized from appropriate precursors such as tin or boron precursors via standard methods such as those described in Jannetti et al, J Nucl Med, 2018, 59:8, 1225-1233; Laird et al, J Thorac Oncol, 2019; Makvandi et al, Mol Cancer Ther, 2019, 18:1195-1204; Pirovano et al, BioRxiv, 2019 and Reilly et al., Org. Lett. 2018, 20, 1752-1755, each of which is incorporated herein by reference in their entireties. Late-stage radiolabeling may be used for labeling PARPi conjugates to maximize radiochemical yield and molar activity. A Valine-Citrulline-PABC linker (5) is attached to the radiolabel precursor (4) to form synthon (6) which can be coupled to an antibody that targets a tumor-associated antigen or a tumor-specific antigen. Linker (5) is composed of four components: 1) an extended spacer (ES) having the structure NH₂—(CH₂)₆—C(O)—; 2) an enzyme cleavable linker (ECL) composed of a dipeptide, valine-citrulline dipeptide; 3) a self-immolative group (SIG), p-aminobenzylalcohol (PABA); and 4) a coupling group (Cg), p-nitrophenyl carbonate, for coupling the radiolabeled olaparib synthon (6) to an antibody. The resulting synthon (6) includes an olaparib moiety coupled to an enzyme cleavable, self-immolative linker, having a coupling group for coupling to an antibody that targets a tumor-associated antigen or a tumor-specific antigen.

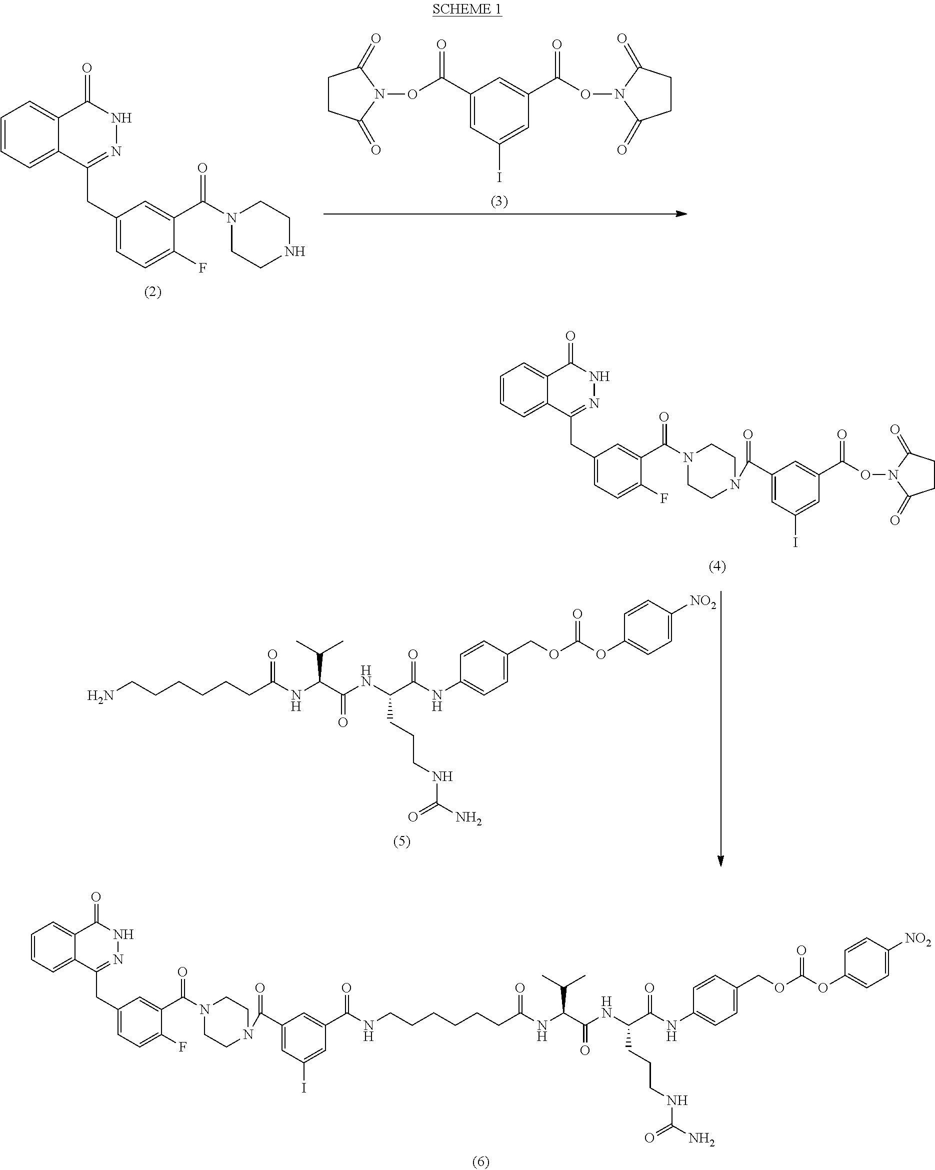 [SMILES: O=C(c1cc(Cc2n[nH]c(=O)c3ccccc23)ccc1F)N1CCNCC1 ; O=C(ON1C(=O)CCC1=O)c1cc(I)cc(C(=O)ON2C(=O)CCC2=O)c1 ; O=C(ON1C(=O)CCC1=O)c1cc(I)cc(C(=O)N2CCN(C(=O)c3cc(Cc4n[nH]c(=O)c5ccccc45)ccc3F)CC2)c1 ; CC(C)[C@H](NC(=O)CCCCCCN)C(=O)N[C@@H](CCCNC(N)=O)C(=O)Nc1ccc(COC(=O)Oc2ccc([N+](=O)[O-])cc2)cc1 ; CC(C)[C@H](NC(=O)CCCCCCNC(=O)c1cc(I)cc(C(=O)N2CCN(C(=O)c3cc(Cc4n[nH]c(=O)c5ccccc45)ccc3F)CC2)c1)C(=O)N[C@@H](CCCNC(N)=O)C(=O)Nc1ccc(COC(=O)Oc2ccc([N+](=O)[O-])cc2)cc1]

### Example 2. Preparation of Olaparib/TTA Synthon With a Disulfide Linker

An iodine containing analogue of olaparib (4) is prepared as described in Example 1. Scheme 2 depicts an example of a method for making a olaparib synthon (9) that incorporates radiolabeled olaparib as a PARP-1 inhibitor, having a disulfide linker, which can be coupled to an antibody that binds to a tumor-associated antigen/receptor or a tumor-specific antigen/receptor. Radiolabel precursor (4), synthesized according to Scheme 1, is reacted with a protected disulfide linker (BocNH—(CH₂)₂—S—S—(CH₂)₂)—NH₂) to allow mono addition of the linker to the precursor (4). 10 The resulting protected disulfide amide (not shown) is deprotected using trifluoroacetic acid to form disulfide intermediate (7). A spacer (—OC(O)—(CH₂)₈—C(O)O—) having N-succinimidyl ester coupling groups (8) is reacted with the disulfide intermediate (7) to form olaparib synthon (9).

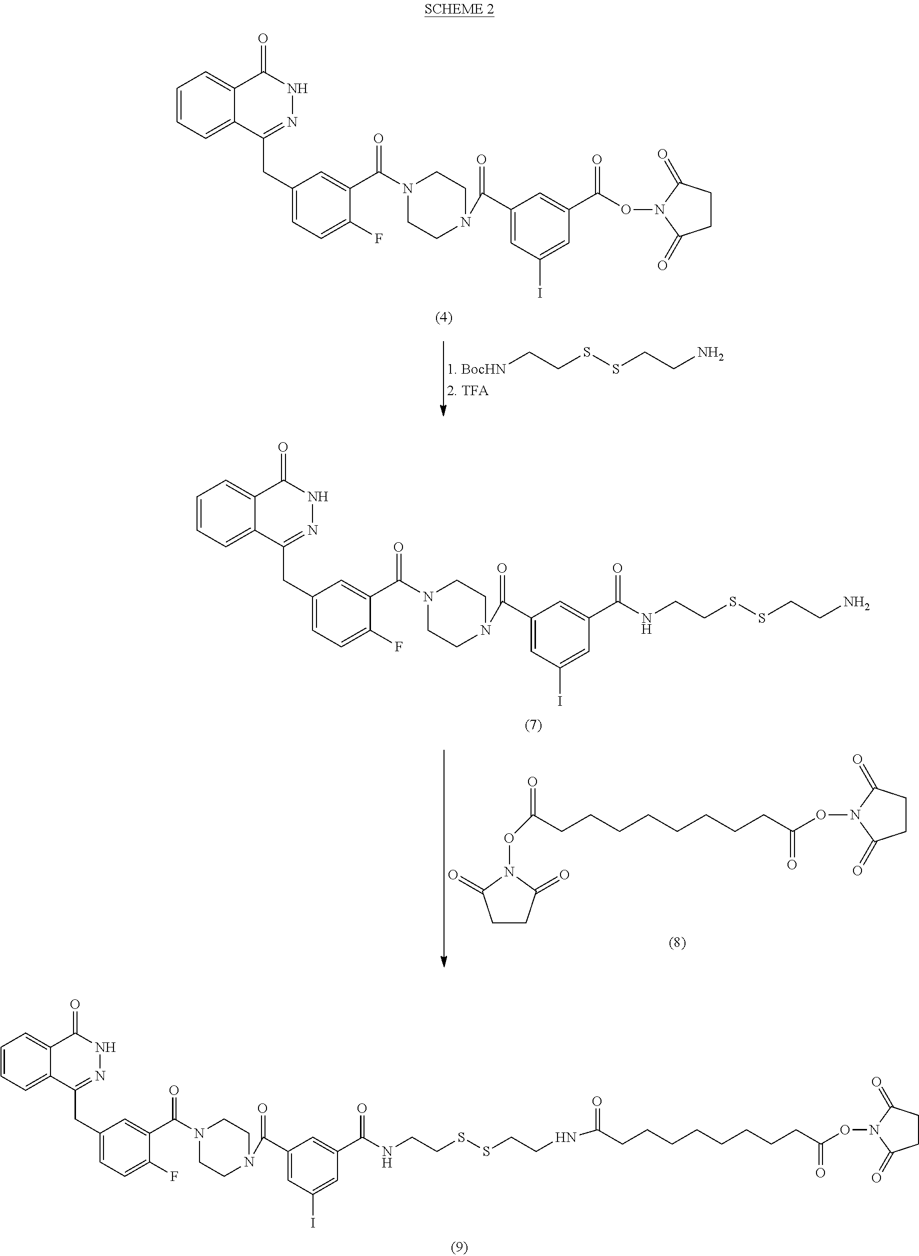

### Example 3. Preparation of Olaparib Conjugate With a PEG Spacer

As depicted in Scheme 3, an analogue of olaparib is prepared by reacting olaparib derivative (2) with radiolabeled precursor moiety (3) to form the intermediate (4). A PEG spacer is coupled to PSMA-binding pharmacophore (10) to form PEG/PSMA pharmacophore-binding moiety (11). PEG/PSMA pharmacophore-binding moiety (11) is reacted with intermediate (4) linking the olaparib moiety to the PEG spacer to form synthon intermediate (12). The “X” group of synthon intermediate (12) is substituted with a radionuclide according to the procedure set forth in the scheme. The resulting radiolabeled olaparib synthon (13) includes an olaparib moiety coupled through a PEG spacer to a tumor targeting agent that targets a tumor-associated antigen or a tumor-specific antigen (e.g., PSMA).

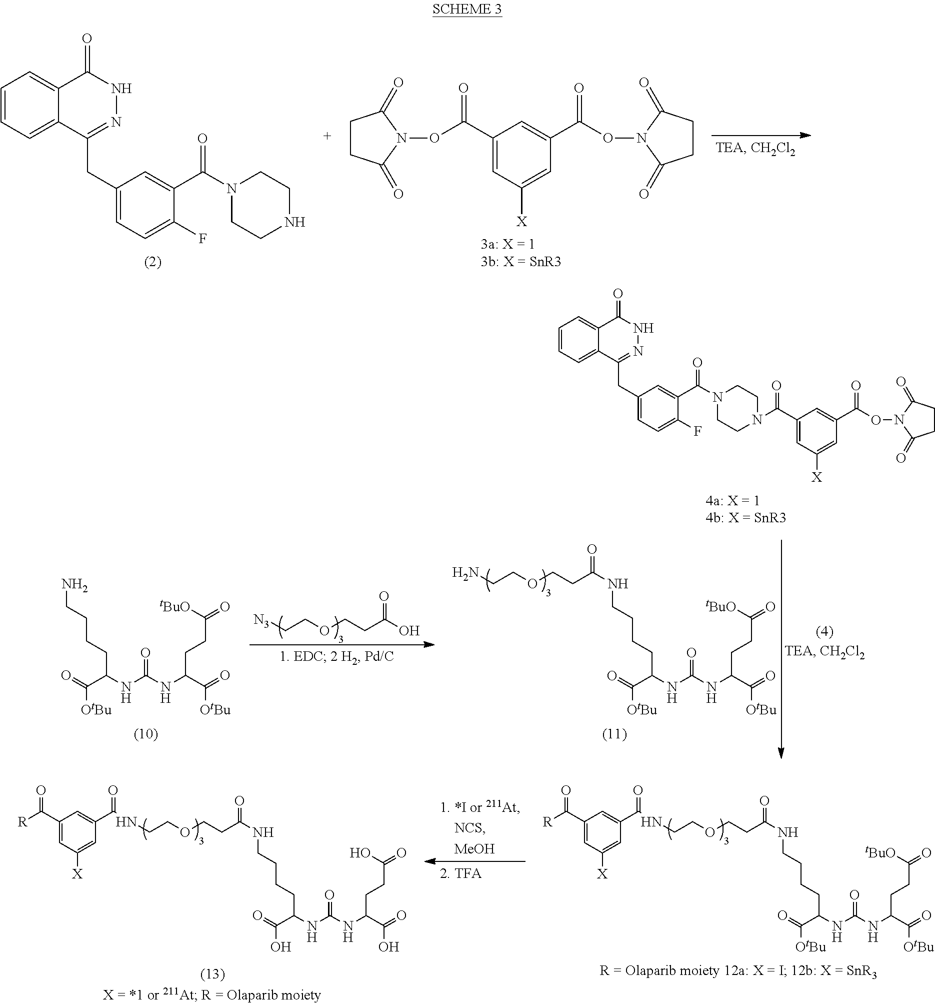

Synthesis of Protected Standard—tri-tert-butyl (20S,24S)-1-(3-(4-(2-fluoro-5-((4-oxo-3,4-dihydrophthalazin-1-yl)methyl)benzoyl)piperazine-1-carbonyl)-5-iodophenyl)-1,14,22-trioxo-5,8,11-trioxa-2,15,21,23-tetraazahexacosane-20,24,26-tricarboxylate (12a)

The intermediate 2,5-dioxopyrrolidin-1-yl-3-(4-(2-fluoro-5-((4-oxo-3,4-dihydrophthalazin-1-yl)methyl)benzoyl)piperazine-1-carbonyl)-5-iodobenzoate (4a) was synthesized by the treatment of one equivalent of bis(2,5-dioxopyrrolidin-1-yl) 5-iodoisophthalate (3a, Vaidyanathan et al., Bioorg. Med. Chem. 2012; 20: 6929-6939) with one equivalent of commercially available 4-(4-fluoro-3-(piperazine-1-carbonyl)benzyl)phthalazin-1(2H)-one (2). Although chromatography was performed, the product contained some acid. A mixture of 4a (200 mg, 1.87 Eq, 271 μmol), tri-tert-butyl (18S,22S)-1-amino-12,20-dioxo-3,6,9-trioxa-13,19,21-triazatetracosane-18,22,24-tricarboxylate (11, 100 mg, 1 Eq, 145 μmol), EDC (83.2 mg, 3.0 Eq, 434 μmol), triethylamine (43.9 mg, 60.5 μL, 3.0 Eq, 434 μmol) in 25 mL dichloromethane was stirred at room temperature for 4 h. Water was added and the organic layer was separated and dried with anhydrous MgSO₄. The volatiles were removed and the crude product was chromatographed using a 10 g BIOTAGE SNAP ULTRA column (DCM:MeOH(9:1) to afford tri-tert-butyl (20S,24S)-1-(3-(4-(2-fluoro-5-((4-oxo-3,4-dihydrophthalazin-1-yl)methyl)benzoyl)piperazine-1-carbonyl)-5-iodophenyl)-1,14,22-trioxo-5,8,11-trioxa-2,15,21,23-tetraazahexacosane-20,24,26-tricarboxylate (12a, 40 mg, 30 μmol, 21%) as an oil. It was further purified using a Biotage SFar 6 g C18 reversed-phase column (100% water to 100% MeCN) to give 18 mg of a pure white solid. LRMS: (M+H)⁺ 1314. HRMS: Calcd for C₆₁H₈₂FIN₈O₁₅ (M+H)⁺: 1313.5001, Found: 1313.4928.

Synthesis of the Tin Precursor, tri-tert-butyl (20S,24S)-1-(3-(4-(2-fluoro-5-((4-oxo-3,4-dihydrophthalazin-1-yl)methyl)benzoyl)piperazine-1-carbonyl)-5-(trimethylstannyl)phenyl)-1,14,22-trioxo-5,8,11-trioxa-2,15,21,23-tetraazahexacosane-20,24,26-tricarboxylate (12a)

The intermediate 2,5-dioxopyrrolidin-1-yl 3-(4-(2-fluoro-5-((4-oxo-3,4-dihydrophthalazin-1-yl)methyl)benzoyl)piperazine-1-carbonyl)-5-(trimethylstannyl)benzoate was synthesized by the treatment of one equivalent of bis(2,5-dioxopyrrolidin-1-yl) 5-(trimethylstannyl)isophthalate (3b) (Vaidyanathan et al., Bioorg. Med. Chem. 2012; 20: 6929-6939) with one equivalent of 4-(4-fluoro-3-(piperazine-1-carbonyl)benzyl)phthalazin-1(2H)-one (2). Although chromatography was performed, the product was contaminated with some hydrolyzed product. A mixture of this compound as such (4b, 175 mg, 5.2 Eq, 226 μmol), tri-tert-butyl (18S,22S)-1-amino-12,20-dioxo-3,6,9-trioxa-13,19,21-triazatetracosane-18,22,24-tricarboxylate (11, 30 mg, 1 Eq, 43 μmol), EDC (8.3 mg, 1 Eq, 43 μmol), triethylamine (4.4 mg, 6.1 μL, 1 Eq, 43 μmol) in 20 mL dichloromethane was stirred at room temperature for 4 h. Water was added and the organic layer was separated and dried over anhydrous MgSO₄. The volatiles were removed and the resultant solid was chromatographed using a Biotage SFar 6 g C18 reversed-phase column (100% water to 100% MeCN) to give tri-tert-butyl (20S,24S)-1-(3-(4-(2-fluoro-5-((4-oxo-3,4-dihydrophthalazin-1-yl)methyl)benzoyl)piperazine-1-carbonyl)-5-(trimethylstannyl)phenyl)-1,14,22-trioxo-5,8,11-trioxa-2,15,21,23-tetraazahexacosane-20,24,26-tricarboxylate (12b, 8.1 mg, 6.0 μmol, 14%) as an off-white foam. LRMS: (M+H)⁺ 1351.0 (Calcd. 1351.2)

Synthesis of the Standard—(20S, 24S)-1-(3-(4-(2-fluoro-5-((4-oxo-3,4-dihydrophthalazin-1-yl)methyl)benzoyl)piperazine-1-carbonyl)-5-iodophenyl)-1,14,22-trioxo-5,8,11-trioxa-2,15,21,23-tetraazahexacosane-20,24,26-tricarboxylic Acid (13)

Trifluoroacetic acid (TFA; 1.0 ml) was added to a solution of tri-tert-butyl 1-(3-(4-(2-fluoro-5-((4-oxo-3,4-dihydrophthalazin-1-yl)methyl)benzoyl)piperazine-1-carbonyl)-5-iodophenyl)-1,14,22-trioxo-5,8,11-trioxa-2,15,21,23-tetraazahexacosane-20,24,26-tricarboxylate (12a, 15 mg, 1 Eq, 11 μmol) in 3.0 ml of dichloromethane. The solution was stirred at room temperature for 16 h and concentrated to dryness. The resulting residue was purified by using a Biotage 6 g SFar C18 reversed-phase column (0.1% TFA in water 100% to 0.1% TFA in MeCN 100%). The fractions containing the product were collected and concentrated under reduced pressure to give (20S,24S)-1-(3-(4-(2-fluoro-5-((4-oxo-3,4-dihydrophthalazin-1-yl)methyl)benzoyl)piperazine-1-carbonyl)-5-iodophenyl)-1,14,22-trioxo-5,8,11-trioxa-2,15,21,23-tetraazahexacosane-20,24,26-tricarboxylic acid (13, 4.2 mg, 3.7 μmol, 32%) as a white solid. LRMS: (M+H)⁺ 1145. HRMS: Calcd for C₄₉H₅₈FIN₈O₁₅ (M+H)⁺: 1145.3123, Found: 1145.3124.

Synthesis of (20S, 24S)-1-(3-(4-(2-fluoro-5-((4-oxo-3,4-dihydrophthalazin-1-yl)methyl)benzoyl)piperazine-1-carbonyl)-5-[¹³¹I]iodophenyl)-1,14,22-trioxo-5,8,11-trioxa-2,15,21,23-tetraazahexacosane-20,24,26-tricarboxylic Acid (13)

A solution of [¹³¹I]iodide in 0.1N NaOH (˜1 μL; 3 mCi) was added to a solution of N-chlorosuccinimide in methanol (25 μL of 2 mg/mL) followed by a solution of the tin precursor (12b) in methanol (10 μL; 10 mg/mL), 10 μL of glacial acetic and 25 μL of methanol. After mixing thoroughly, the mixture was heated at 37° C. for 20 min. The volatiles were evaporated with a gentle stream of argon, 100 μL of trifluoroacetic acid (TFA) was added to the residual activity and the mixture heated at 37° C. for 10 min. TFA was evaporated with a gentle stream of argon, the residual activity reconstituted in 100 μL of 5% (v/v) acetonitrile in water containing 0.1% TFA and injected onto a reversed-phase analytical HPLC column (Agilent Poroshell 120 C18, 2.7 μm, 4.6×50 mm). The column was eluted with a gradient consisting of 0.1% TFA in each water (A) and acetonitrile (B) and linearly increasing proportion of B from 5% to 95% in 15 min. Under these conditions, the final deprotected product (13) elutes with a retention time of ˜7 min. HPLC fractions containing the product were pooled and most of the acetonitrile was evaporated with a stream of argon, diluted with water (10 mL) and passed through an activated C18 SeptemberPak cartridge. The cartridge was washed with 10 mL water and the product eluted with MeOH (2×100 μL). MeOH was evaporated and the product reconstituted in PBS for biological assays. Radiochemical yield was 57%.

Uptake and Internalization of (20S,24S)-1-(3-(4-(2-fluoro-5-((4-oxo-3,4-dihydrophthalazin-1-yl)methyl)benzoyl)piperazine-1-carbonyl)-5-[¹³¹I]iodophenyl)-1,14,22-trioxo-5,8,11-trioxa-2,15,21,23-tetraazahexacosane-20,24,26-tricarboxylic Acid ([¹³¹I]PARP-PSMA Combo Agent 13) in PSMA⁺ PIP Cells

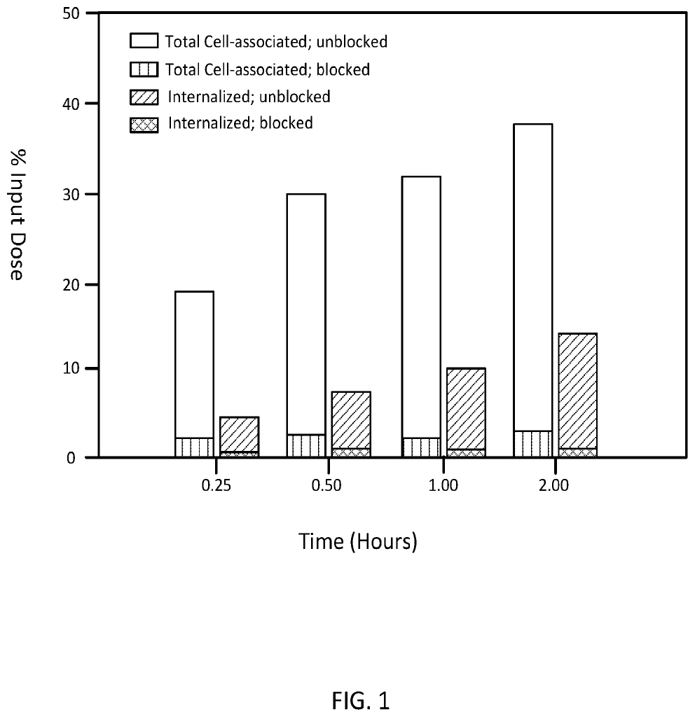

PSMA-specific uptake and internalization of [¹³¹I]PARP-PSMA combo agent 13 was determined by incubation with 5×10⁵ PIP cells in triplicate in the absence or presence of 200 μM 2-PMPA at 37° C. for 0.25-2 h. After removing the unbound activity-containing medium, the cells were washed with 50 mM glycine, pH 2.8 and finally, cells were lysed with 1M NaOH. Radioactivity in the supernatants, glycine wash (membrane-bound) and lysate (internalized) fractions was counted and percentage of input dose calculated. FIG. 1 shows the results for total cell-associated and internalized activity as a function of time. Total cell-associated activity was 18.7±1.0%, 30.0±0.9%, 31.8±1.0 and 37.9±0.6% at 15, 30, 60, and 120 min, respectively. 2-PMPA blocked uptake by >80% suggesting PSMA-specific uptake. The total cell-associated activity reported (Kiess et al., J Nucl Med, 2016; 57: 1569-1575. DOI: 10.2967/jnumed. 116.174300) for labeled PSMA inhibitor [¹³¹I]DCIBzL in PIP cells (5.0±0.3%, 9.3±1.5% and 16.8±1.0% at 30, 60, and 120 min, respectively) was considerably lower suggesting that the new combination agent exhibited a higher level of PSMA-specific uptake.

Subcellular Fractionation of (20S,24S)-1-(3-(4-(2-fluoro-5-((4-oxo-3,4-dihydrophthalazin-1-yl)methyl) benzoyl)piperazine-1-carbonyl)-5-[¹³¹I]iodophenyl)-1,14,22-trioxo-5,8,11-trioxa-2,15,21,23-tetraazahexacosane-20,24,26-tricarboxylic acid ([¹³¹I]PARP-PSMA Combo Agent 13) in PSMA⁺ PIP Cells

Subcellular fractionation of [¹³¹I]PARP-PSMA combo agent 13 in PSMA+ PIP cells was determined using The Subcellular Protein Fractionation Kit for Cultured Cells (Number 78840) purchased from Thermo Scientific. PIP Cells (5 million) were incubated with MBq of [¹³¹I] 13 in 1.5 ml tubes with a final incubation volume of 500 μL. Stepwise separation and preparation of cytoplasmic, membrane, nuclear soluble, chromatin-bound and cytoskeletal protein extracts from PSMA⁺ PIP cells were achieved by following the protocol provided by Thermo Scientific. The following groups of experiments were performed:
1. ([¹³¹I]PARP-PSMA combo agent 13.
2. ([¹³¹I]PARP-PSMA combo agent 13+2-PMPA blocking agent (680 μM)
3. ([¹³¹I]PARP-PSMA combo agent 13+Olaparib blocking agent (6.8 mM)
4. ([¹³¹I]PARP-PSMA combo agent 13+2-PMPA blocking agent (6.8 mM)+Olaparib blocking agent (6.8 mM)

Table 1 shows the subcellular fractionation results for above 4 groups. The total cellular uptake of ([¹³¹I]PARP-PSMA combo agent 13 was 26.69% after 1 hour incubation. A majority of cellular uptake (20.70%) was on the membrane and the nuclear soluble portion was 2.17%. The blocking agents 2-PMPA and Olaparib substantially decreased cellular uptake. The total cellular uptake decreased from 26.69% for the test without blocking agents to 4.99% for the group with PMPA blocking agent, 13.44% for the group with Olaparib blocking agent, and 3.78% for the group with both PMPA and Olaparib blocking agent. Similarly, the membrane-bound radioactivity decreased from 20.70% for the test without blocking agents to 3.23% for the group with PMPA blocking agent, 9.00% for the group with Olaparib blocking agent, and 1.44% for the group with both PMPA and Olaparib blocking agents.

TABLE 1

| | | | | PMPA + |
|---|---|---|---|---|
| 1 h time point (%) | PSMA-PARP | PMPA | Olaparib | Olaparib |
| Supernatant (Wash Out) | 73.31 | 86.85 | 82.42 | 87.21 |
| Cytoplasmic | 2.50 | 1.47 | 3.09 | 2.13 |
| Membrane | 20.70 | 3.23 | 9.00 | 1.44 |
| Nuclear Soluble | 2.17 | 0.11 | 1.01 | 0.14 |
| Micrococcal Nuclease | 0.59 | 0.09 | 0.14 | 0.03 |
| Cytoskeletal Protein | 0.28 | 0.03 | 0.15 | 0.02 |
| Cells Left Over | 0.45 | 0.06 | 0.07 | 0.02 |
| Total Cellular Uptake | 26.69 | 4.99 | 13.44 | 3.78 |

### Example 4. Preparation of Olaparib Synthon With a PEG Spacer and Disulfide Linker

An iodine containing derivative of olaparib (4a) is prepared as described in Example 1. Referring to Scheme 4, a disulfide linker is coupled to olaparib derivative (4a) by reacting olaparib derivative (4a) with tert-butyl (2-((2-aminoethyl)disulfaneyl)ethyl)carbamate (14) to form the olaparib disulfide moiety (15). A PEG spacer (16) is coupled to olaparib disulfide moiety (15) to form olaparib PEG/disulfide moiety (17). PSMA-binding pharmacophore (10) (See Scheme 3) is coupled to olaparib PEG/disulfide moiety (17) to produce protected olaparib synthon (18), which is deprotected to form olaparib synthon (19). The resulting olaparib synthon (19) includes an olaparib moiety coupled to a disulfide linker and a PEG spacer attached to a tumor targeting agent that targets a tumor-associated antigen or a tumor-specific antigen (e.g., PSMA).

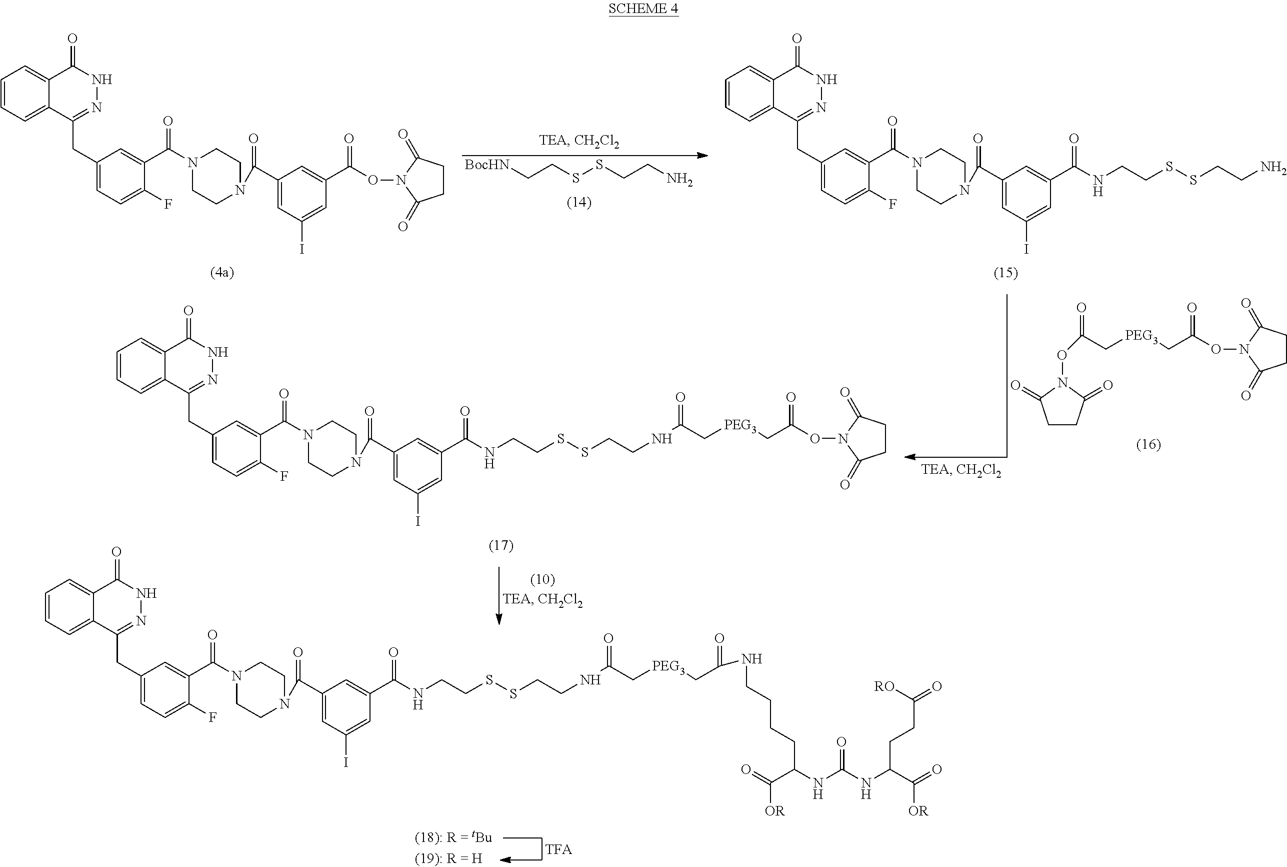

### Example 5. Alternate Preparation of Olaparib Synthon With a PEG Spacer and Disulfide Linker

Scheme 5 depicts an alternate synthetic route to olaparib synthon with a PEG spacer and disulfide linker. In this scheme the spacer/PSMA-binding pharmacophore portion of the olaparib synthon is synthesized first. PSMA-binding pharmacophore (10) serves as the starting material for Scheme 5. It is coupled to a PEG spacer to give the PSMA-binding pharmacophore-spacer intermediate (20). A disulfide linker is coupled to the intermediate (20) to complete the PSMA-binding pharmacophore/spacer (21). An iodine or tin containing derivative of olaparib (4a) is constructed onto the linker/spacer (21). For this, first an iodobenzene or trialkylstannylbenzene moiety-containing molecule (3a/3b) having two coupling groups (e.g., succinimidyloxycarbonyl) is coupled to spacer/PSMA-binding pharmacophore (21) to form intermediate (22). Intermediate (22) is reacted with olaparib derivative (2) to form synthon (23a). Olaparib synthon (23a) can be deprotected using trifluoroacetic acid. The resulting deprotected synthon (23b) includes an olaparib moiety coupled to a disulfide linker and a PEG spacer. Olaparib synthon (23a or 23b) targets a tumor-associated antigen or a tumor-specific antigen (e.g., PSMA).

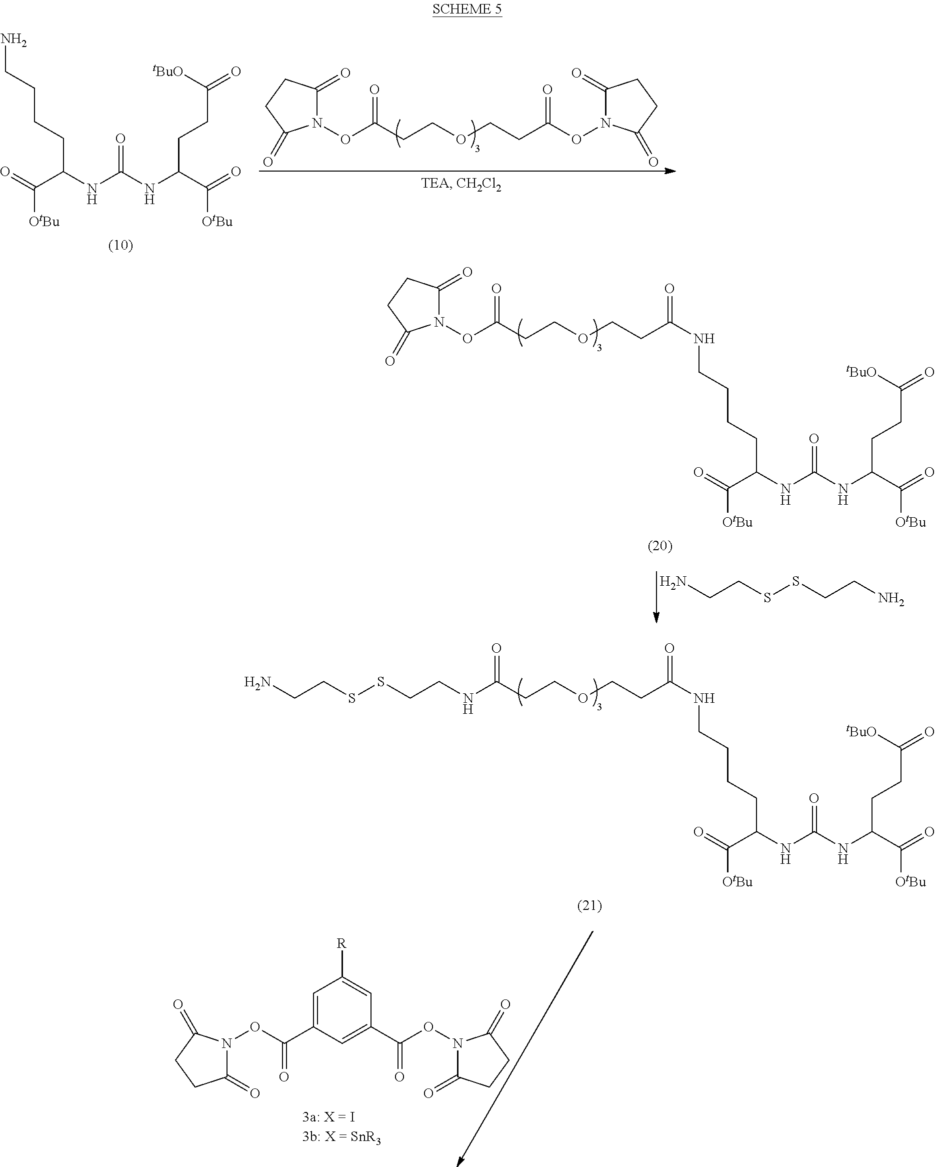

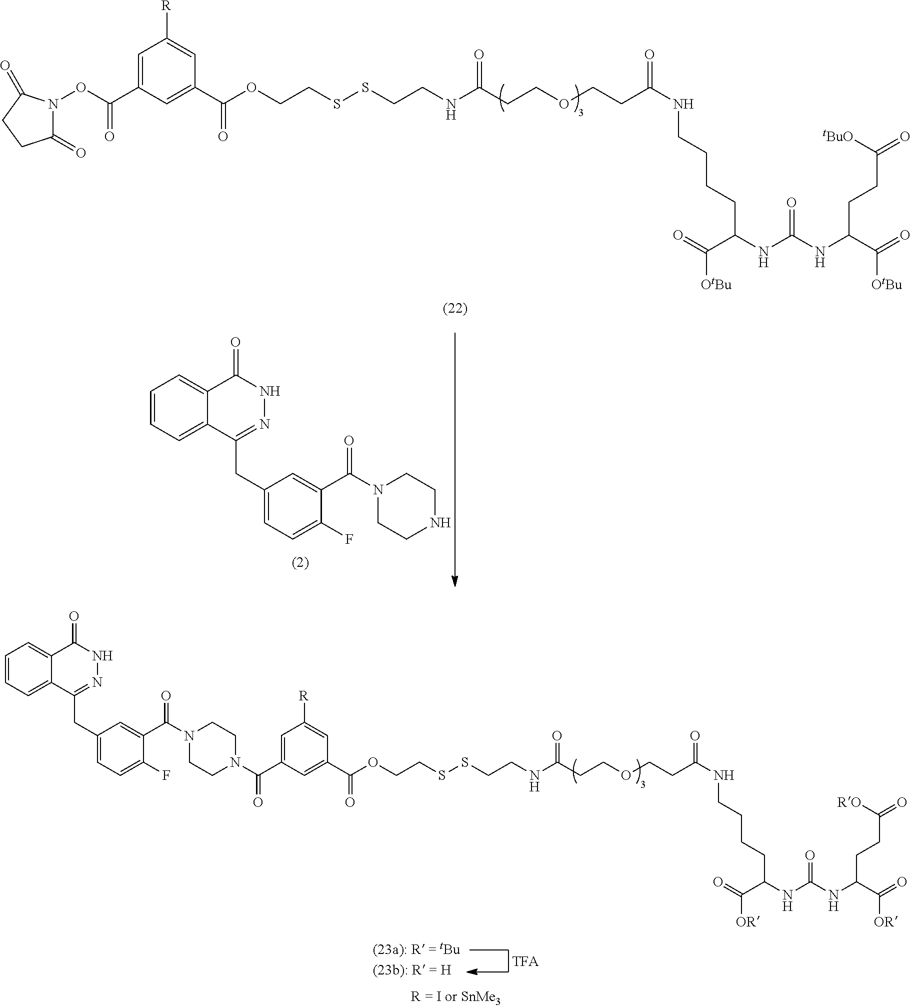

### Example 6-Olaparib Synthon for sdAb Site-Specific Conjugation

In an embodiment, an olaparib-containing synthon has the structure (24).

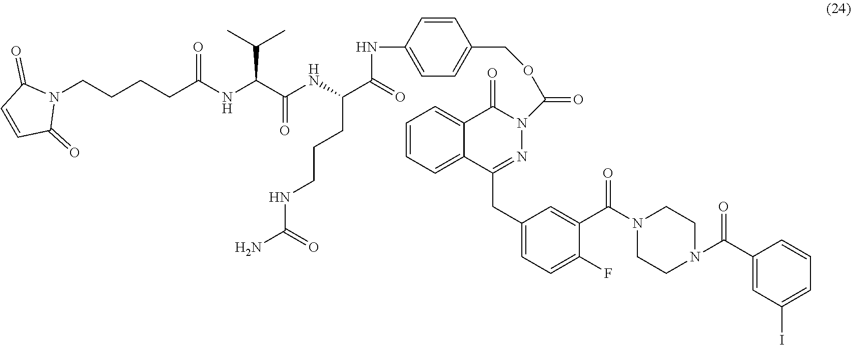 [SMILES: CC(C)[C@H](NC(=O)CCCCN1C(=O)C=CC1=O)C(=O)N[C@@H](CCCNC(N)=O)C(=O)Nc1ccc(COC(=O)n2nc(Cc3ccc(F)c(C(=O)N4CCN(C(=O)c5cccc(I)c5)CC4)c3)c3ccccc3c2=O)cc1]

Synthon (24) includes a maleimide group for coupling to a cysteine group of an antibody (e.g., a sdAb). Iodine group can be an iodine radionuclide (e.g., ¹³¹I). Synthesis of olaparib synthon (24) is shown in Schemes 6-9.

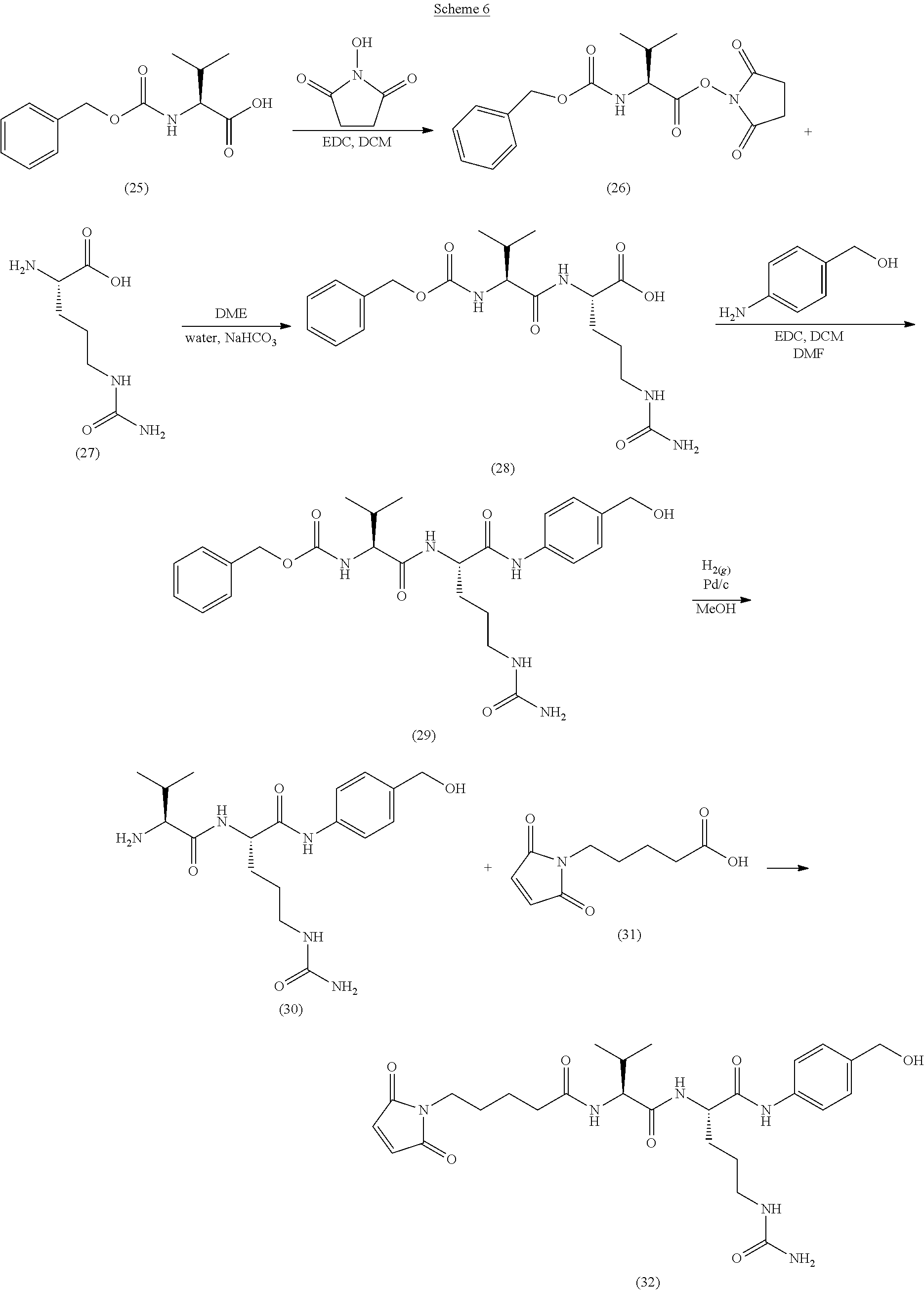

Scheme 6 depicts the synthesis of the self immolative-enzyme cleavable group (32) of the conjugate. The self immolative-enzyme cleavable group (32) of the conjugate includes a maleimide group, for coupling to a tumor targeting agent (e.g., an antibody), coupled to a mc-vc-PABA self immolative linker. Staring with a benzyl carbamate protected valine (25), the carboxylic acid is activated by reaction with N-hydroxysuccinimide. The activated intermediate product (26) is reacted with citrulline (27) to form the Val-Cit portion (28) of the linker. The Val-Cit portion is reacted with p-aminobenzyl alcohol to form the vc-PABA portion (29) of the linker (32). The benzyl carbamate protecting group is removed from the Val on intermediate (29) and the unprotected Val reacted with maleimidocaproyic acid (31) to form self immolative enzyme cleavable group (32).

Scheme 7 shows the synthesis of the modified olaparib portion (35) of the olaparib synthon (24). Olaparib is altered by hydrolysis of cyclopropane carboxamide , which frees the nitrogen on the piperazine ring, giving olaparib derivative (2). Reaction of olaparib derivative (2) with 4-tri-n-butylstannyl benzoic acid gives the modified olaparib portion (35) of the olaparib synthon (24). The tin substituent serves as a handle for the introduction of a radionuclide onto the conjugate.

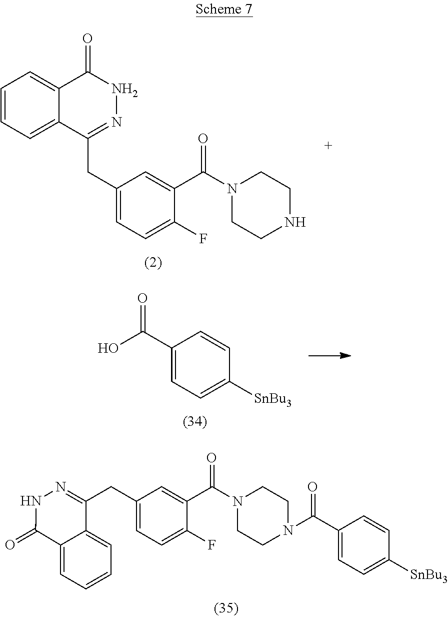

Scheme 8 shows the attachment of a carbonate coupling agent (36) to the self immolative-enzyme cleavable group (32) to give Linker (37).

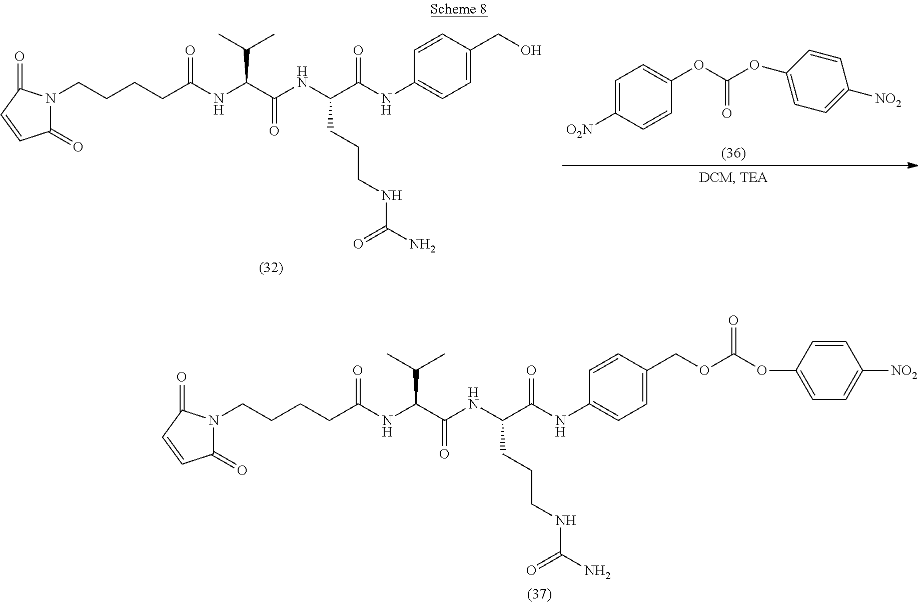 [SMILES: CC(C)[C@H](NC(=O)CCCCN1C(=O)C=CC1=O)C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1 ; CC(C)[C@H](NC(=O)CCCCN1C(=O)C=CC1=O)C(=O)N[C@@H](CCCNC(N)=O)C(=O)Nc1ccc(COC(=O)Oc2ccc([N+](=O)[O-])cc2)cc1]

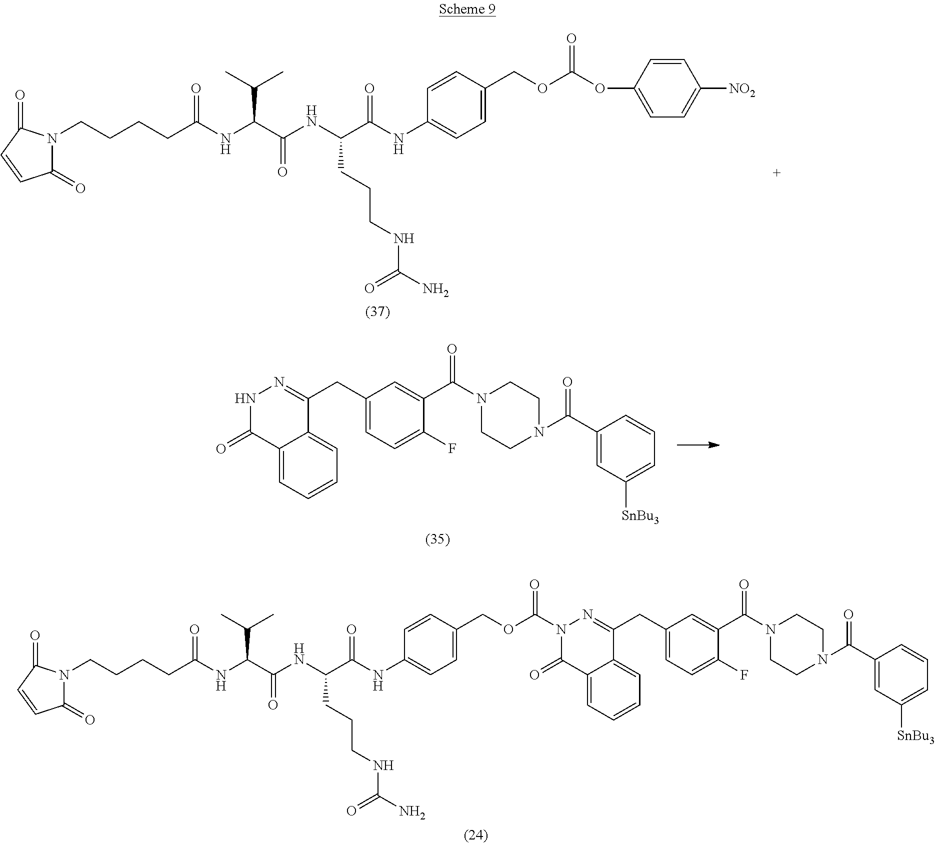

Scheme 9 shows the final assembly of the synthon (24). Linker (37) is reacted with modified olaparib (35) to form the olaparib synthon (24). Olaparib synthon (24) can be reacted with a suitable tumor targeting agent (e.g., an antibody) and the tin substituent replaced with a radionuclide to form an olaparib-antibody conjugate.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A composition comprising a radiolabeled poly(ADP-ribose) polymerase-1 inhibitor (PARPi(Rd)) conjugate, wherein the conjugate comprises PARPi(Rd) coupled to a tumor targeting agent (TTA).

2. The composition of claim 1, wherein the PARPi conjugate comprises Formula I:
PARPi(Rd)-L-TTA (I)
wherein Rd is a radiolabel and PARPi(Rd) is a radiolabeled PARPi,
wherein L is a linker and TTA is a tumor targeting agent, and
wherein the L couples the PARPi(Rd) to the TTA.

3. The composition of claim 1 or 2, wherein the PARPi is a PARP-1 inhibitor.

4. The composition of any one of claims 1-3, wherein the PARPi is selected from the group consisting of olaparib, veliparib, rucaparib, niraparib, pamiparib, and talazoparib.

5. The composition of any one of claims 1-4, wherein the PARPi increases PARP-1 binding affinity to DNA compared to the binding affinity to DNA of uninhibited PARP-1.

6. The composition of any one of claims 1-5, wherein the PARPi slows the release of PARP-1 from DNA compared to the release of uninhibited PARP-1 from DNA.

7. The composition of any one of claims 1-6, wherein the TTA is a single domain antibody fragment (sdAb).

8. The composition of any one of claims 1-6, wherein the TTA is a prostate specific membrane antigen (PSMA) inhibitor/ligand.

9. The composition of any one of claims 1-8, wherein the Rd comprises a radionuclide that emits short-range radiation.

10. The composition of any one of claims 1-8, wherein the Rd decays by the emission of Auger electrons and/or alpha particles.

11. The composition of any one of claims 2-10, wherein the Rd has the structure:
Cg¹-ArQ-Cg²;
wherein Cg¹ is a coupling group that couples ArQ to the PARPi, and Cg² is a coupling group that couples Rd to the L;
wherein Cg¹ and Cg² are each, independently, succinimidyloxycarbonyl, maleimide, —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C —; —C—; —N═; ═N—; —C═N—; —N—C—; —O—N═; ═N—O—; —C═N—O—; —O—N═C—; —N(R¹)—N═; —N—N(R¹)—; —N(R¹)—N═C—; or —C═N—N(R¹)—; or
Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein Ar is an aryl, a heteroaryl, or a radionuclide chelating agent; and
wherein Q is radionuclide.

12. The composition of any one of claims 2-10, wherein the L has the structure:
ES¹-SS-ES²
wherein ES¹ has the structure: Cg³-SP-;
wherein SS is a disulfide bond;
wherein ES² has the structure: -SP-Cg⁴-;
wherein Cg³ and Cg⁴ are each, independently, succinimidyloxycarbonyl; maleimide;
—OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═;═N—N(R¹)—; —N(R¹)—N═C—; or —C—N—N(R¹)—; or Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N═C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein SP is —C₁-C₁₀ alkylene-; —C₁-C₁₀ alkenylene-; —C₁-C₁₀ alkynylene-; carbocyclo-; —O—(C₁-C₈ alkylene)-; —O—(C₁-C₈ alkenylene)-; —O—(C₁-C₈ alkynylene)-; -arylene-, —C₁-C₁₀ alkylene-arylene-; —C₂-C₁₀ alkenylene-arylene-; —C₂-C₁₀ alkynylene-arylene; -arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—;
wherein r is an integer ranging from 1-10;
wherein Cg³ is a coupling group that forms a covalent bond to the Rd; and wherein Cg⁴ is a coupling group that forms a covalent bond to the TTA.

13. The composition of any one of claims 2-10, wherein the L has the structure:
ES-ECL-SIG
wherein ES has the structure: Cg³-SP-Cg⁴;
Cg³ and Cg⁴ are each, independently, succinimidyloxycarbonyl, maleimide, —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N═C—; —O—N═; ═N—O—; —C═N—O—; —O—N—C—; —N(R¹)—N═; ═N—N(R¹)—; —N(R¹)—N═C—; or —C═N—N(R¹)—; or Cg¹ and Cg² are each, independently, a phenyl group having two functional groups selected from the group consisting of —OC(O)—; —C(O)O—; —OC(O)O—; —OC(O)N(R¹)—; —N(R¹)C(O)—; —C(O)N(R¹)—; —N(R¹)C(O)O—; —N(R¹)C(O)N(R²)—; —C(O)—; —OC(R¹)(R²)—; —C(R¹)(R²)O—; —OC(R¹)(R²)O—; —C(R¹)(R²)—; —S—; —S—S—; C—; ═C—; —N═; ═N—; —C═N—; —N—C—; —O—N═; ═N—O—; —C═N—O—; —O—N—C—; —N(R¹)—N═; ═N—N(R¹)—; —N(R¹)—N═C—; and —C═N—N(R¹)—;
wherein R¹ and R² are independently selected from H, branched or unbranched C₁₋₁₀ alkyl, or aryl;
wherein Cg³ is a coupling group that forms a covalent bond to the Rd component and Cg⁴ is a coupling group that forms a covalent bond to ECL;
wherein SP is —C₁-C₁₀ alkylene-; —C₁-C₁₀ alkenylene-; —C₁-C₁₀ alkynylene-; -carbocyclo-; —O—(C₁-C₈ alkylene)-; —O-(C₁-C₈ alkenylene)-; —O—(C₁-C₈ alkynylene)-; -arylene-; —C₁-C₁₀ alkylene-arylene-; —C₂-C₁₀ alkenylene-arylene; —C₂-C₁₀ alkynylene-arylene; arylene-C₁-C₁₀ alkylene-; -arylene-C₂-C₁₀ alkenylene-; -arylene-C₂-C₁₀ alkynylene-; —C₁-C₁₀ alkylene-(carbocyclo)-; —C₂-C₁₀ alkenylene-(carbocyclo)-; —C₂-C₁₀ alkynylene-(carbocyclo)-; -(carbocyclo)-C₁-C₁₀ alkylene-; -(carbocyclo)-C₂-C₁₀ alkenylene-; -(carbocyclo)-C₂-C₁₀ alkynylene; -heterocyclo-; —C₁-C₁₀ alkylene-(heterocyclo)-; —C₂-C₁₀ alkenylene-(heterocyclo)-; —C₂-C₁₀ alkynylene-(heterocyclo)-; -(heterocyclo)-C₁-C₁₀ alkylene-; -(heterocyclo)-C₂-C₁₀ alkenylene-; -(heterocyclo)-C₁-C₁₀ alkynylene-; —(CH₂CH₂O)ᵣ—; or —(CH₂CH₂O)ᵣ—CH₂—; wherein r is an integer ranging from 1-10;
wherein ECL is an enzyme cleavable linker; and
wherein SIG is a self-immolative group that forms a covalent bond between the ECL and the TTA, wherein upon cleavage of the CL, the SIG decomposes to release the TTA from the ECL.

14. The composition of claim 13, wherein the ECL is cleavable by a lysosomal enzyme.

15. The composition of claim 14, wherein the lysosomal enzyme is a lysosomal protease.

16. The composition of any one of claims 13 to 15, wherein the ECL is a peptide composed of two to four amino acids.

17. The composition of claim 14, wherein the lysosomal enzyme is lysosomal glycosidase.

18. The composition of any one of claims 1-17 wherein the PARPi comprises olaparib.

19. The composition of any one of claims 1-17, wherein the PARPi comprises talazaporib.

20. The composition of claim 1, wherein the PARPi(Rd)-L has the structure (VI): 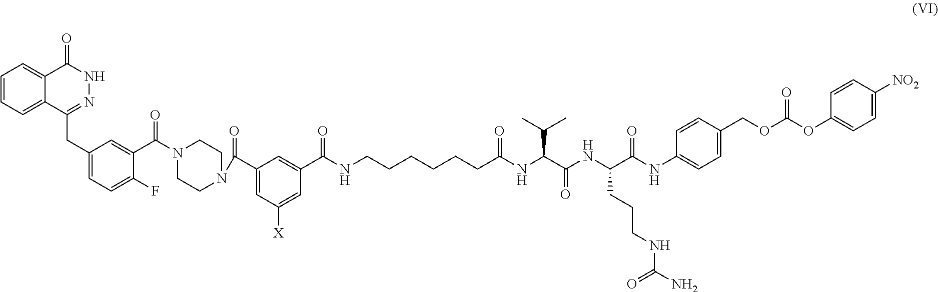
where X is a radionuclide.

21. The composition of claim 1, wherein the PARPi(Rd)-L has the structure (IX): 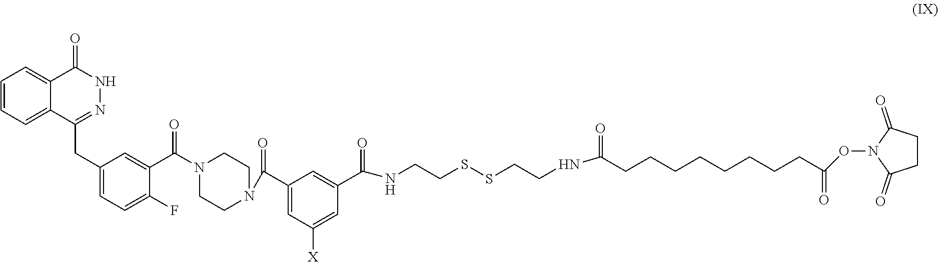
where X is a radionuclide.

22. The composition of claim 1, wherein the PARPi(Rd)-L has the structure: 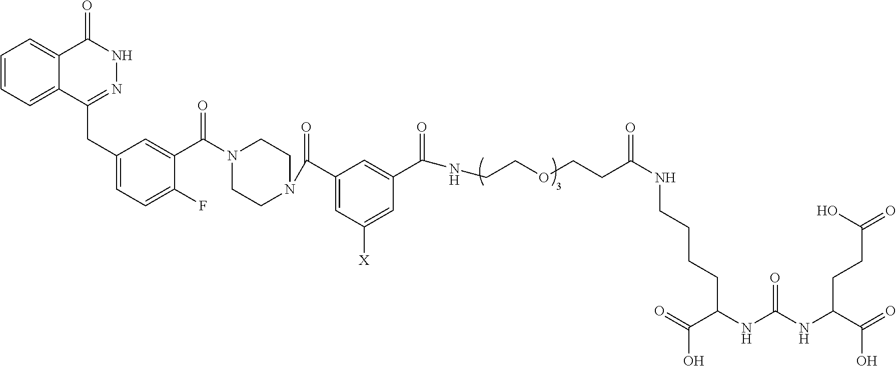
where X is a radionuclide.

23. The composition of claim 1, wherein the PARPi(Rd)-L has the structure: 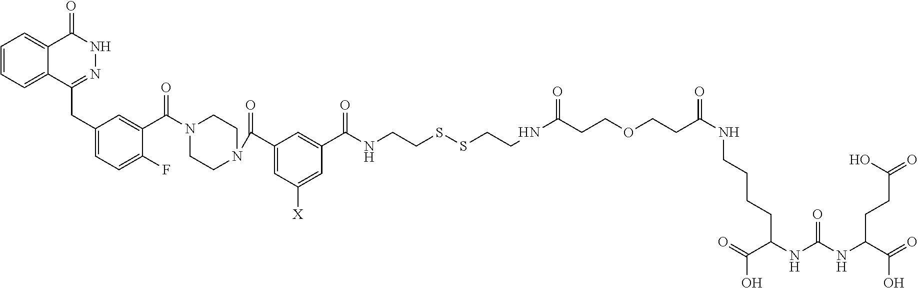
where X is a radionuclide.

24. A composition comprising a poly(ADP-ribose) polymerase-1 inhibitor (PARPi) conjugate, wherein the conjugate comprises a PARPi coupled to a tumor targeting agent (TTA).

25. The composition of claim 24, wherein the PARPi conjugate comprises Formula II:
PARPi-L-TTA; (II)
wherein L is a linker and TTA is a tumor targeting agent, and
wherein the L couples the PARPi to the TTA.

26. A pharmaceutical composition comprising the composition of any one of claims 1-25 and a pharmaceutically acceptable carrier.

27. The pharmaceutical composition of claim 26, further comprising a photosensitizing agent.

28. Use of the pharmaceutical composition of claim 26 or 27 for cancer therapy.

29. A method of radiosensitizing a tumor in a cancer subject comprising administering to the subject the pharmaceutical composition of claim 26 or 27.

30. A method of treating cancer in a subject comprising the steps of (i) administering to the subject the pharmaceutical composition of claim 26 or 27, and (ii) administering ionizing radiation.

31. A method of treating cancer in a subject, comprising administering to the subject the pharmaceutical composition of claim 26 or 27.

32. The method of any one of claims 29-31, wherein the cancer is ovarian cancer.

33. The method of any one of claims 29-31, wherein the cancer is breast cancer.

34. The method of any one of claims 29-31, wherein the cancer is brain cancer.

35. The method of any one of claims 29-31, wherein the cancer is prostate cancer.

36. The method of any one of claims 29-35, wherein the subject has a deficiency in at least one gene involved in the homologous recombination repair (HRR) pathway.

37. The method of any one of claims 29-36, wherein the pharmaceutical composition is administered in combination with chemotherapy.
